(19) United States
(12) Patent Application Publication
(10) Pub. No.: US 2022/0341939 A1
(43) Pub. Date: Oct. 27, 2022

(54) PREDICTIVE TEST FOR IDENTIFICATION OF EARLY STAGE NSCLC STAGE PATIENTS AT HIGH RISK OF RECURRENCE AFTER SURGERY

(71) Applicant: BIODESIX, INC., Boulder, CO (US)

(72) Inventors: Heinrich RODER, Steamboat Springs, CO (US); Joanna RÖDER, Steamboat Springs, CO (US); Lelia NET, Boulder, CO (US); Laura MAGUIRE, Boulder, CO (US)

(73) Assignee: BIODESIX, INC., Boulder, CO (US)

(21) Appl. No.: 17/430,998

(22) PCT Filed: Jan. 29, 2020
(86) PCT No.: PCT/US2020/015626

§ 371 Date: Aug. 13, 2021

Related U.S. Application Data
(60) Provisional application No. 62/806,254, filed on Feb. 15, 2019.

(51) Int. Cl. G01N 33/68; G01N 33/574; G16B 40/20

(52) U.S. Cl. CPC G01N 33/6848; G01N 33/57423; G16B 40/20

(57) ABSTRACT

A method for predicting whether an early stage (IA, IB) non-small-cell lung cancer (NSCLC) patient is at a high risk of recurrence of the cancer following surgery involves subjecting a blood-based sample from the patient (obtained prior to, at, or after the surgery) to mass spectrometry and classification with a computer implementing a classifier. If the patients blood sample is classified as “high risk”, highest risk“or the equivalent, the patient can be guided to more aggressive treatment post-surgery. The classifier, or combination of classifiers, can be arranged in a hierarchical manner to make intermediate classifications, such as intermediate/high or intermediate/low, as well as low risk” or “lowest risk” classifications. Such additional classifications may guide clinical decisions as well.

### PRIORITY

This application claims priority benefits of U.S. provisional application serial no. 62/806,254 filed Feb. 15, 2019, the content of which is incorporated by reference herein.

### FIELD

This document describes a practical blood-based test for determining whether an early stage non-small-cell lung cancer (NSCLC) patient is likely to have a high risk of recurrence of cancer after surgery to remove the cancer. The test can be performed at, before, and/or after the time of surgery. Where the test determines that the patient is at a high risk of recurrence of the cancer it indicates that the patient should be considered for more aggressive treatment, such as adjuvant chemotherapy or radiation in addition to the surgery.

### BACKGROUND

The majority of cancer deaths in the United States are due to lung cancer. It is estimated that there were in excess of 200,000 new cases diagnosed and more than 150,000 lung cancer deaths in 2018. See https://seer.cancer.gov/statfacts/html/lungb.html. Approximately 80-85% of lung cancers are non-small cell lung cancer (NSCLC). See https://www.cancer.org/cancer/non-small-cell-lung-cancer/about/what-is-non-small-cell-lung-cancer.html. Currently, around 16% of lung cancers are diagnosed as localized disease. However, this proportion may increase in the future as lung cancer screening programs gain wider adoption.

Patients with Stage 1 disease are generally treated with surgical resection, although radiotherapy is recommended for patients who are inoperable or refuse surgery. National Comprehensive Cancer Network (NCCN) Clinical Practice Guidelines in Oncology (NCCN Guidelines) Non-Small Cell Lung Cancer, Version 3.2019-Jan. 18, 2019, Adjuvant therapy is currently not recommended in the NCCN guidelines for Stage IA disease. It is recommended that positive margins from surgery be followed by re-resection (preferred) or by radiotherapy. Observation is indicated as follow up for Stage IA with negative margins. NCCN recommended follow up for Stage IB (and Stage IIA) disease with negative margins from surgery is observation, or chemotherapy for high-risk patients. Factors that indicate high risk include poorly differentiated tumors, vascular invasion, wedge resection, tumor size >4 cm, visceral pleural involvement and unknown lymph node status. Positive margins in surgery for Stage IB and Stage IIA disease call for re-resection (preferred) or radiotherapy, with or without adjuvant chemotherapy. It is recommended that if radiotherapy is given for Stage IIA disease with positive margins, it should be accompanied by adjuvant chemotherapy.

Prognosis for Stage I patients varies from a 5-year survival rate of 92% for Stage IA1 and 83% for Stage IA2 to 77% for Stage IA3, See https://www,cancer.org/cancer/non-small-cell-lung-cancer/detection-diagnosis-staging/survival-rates.html. Five-year survival for patients with Stage IB disease is about 68%, Id.

Hence, although many patients may be cured by surgical intervention, a significant proportion of patients recur. If it were possible to identify the patients with early stage NSCLC at highest risk of recurrence, it may potentially be advantageous for their survival to treat them more aggressively. It is of note, however, that the Lung Adjuvant Cisplatin Evaluation meta-analysis contraindicated adjuvant chemotherapy in the general stage IA population by indicating potentially worse outcomes with adjuvant chemotherapy than without. J-P. Pignon et al, “Lung Adjuvant Cisplatin Evaluation: A Pooled Analysis by the LACE Collaborative Group,” J ClinOncol, pp, 3552-3559, 2008, Therefore, accurate identification of patients at highest risk of recurrence is essential before advocating more aggressive therapies,

Currently, there is no validated test able to reliably identify patients at highest risk of lung cancer recurrence either from tissue collected at surgery or from blood-based samples. Here, we describe a test, based on mass spectrometry of serum collected from patients at or prior to surgery, able to stratify patients by risk of recurrence.

### SUMMARY

In one aspect, a method for performing a risk assessment of recurrence of cancer in an early stage non-small-cell lung cancer patient is described. The method includes a step of performing mass spectrometry on a blood-based sample obtained from the patient and obtaining mass spectrometry data. The method further includes the step of, in a computing machine, performing a hierarchical classification procedure on the mass spectrometry data. In particular, the computing machine implements a hierarchical classifier schema including a first classifier (Classifier A in the following description) producing a class label in the form of high risk or low risk or the equivalent. The class label of “high risk” indicates that the patient providing the sample is at high risk of recurrence of the cancer after surgery, whereas the class label “low risk” indicates that the patient providing the sample is at a relatively low risk of recurrence. In one possible embodiment, if the Classifier A produces the high risk label the sample is classified by a second classifier (Classifier B in the following description) generating a classification label of highest risk or high/intermediate risk or the equivalent. If Classifier B produces the label of highest risk or the equivalent the patient is likewise predicted to have a high risk of recurrence of the cancer following surgery.

In one configuration, the computing machine implements a hierarchical classifier schema including a third classifier (Classifier C in the discussion below) wherein if the Classifier A produces a “low risk” classification label the sample is classified by the third Classifier C and wherein classifier C produces a class label of lowest risk or low/intermediate risk, or the equivalent.

In one configuration, the computing machine stores a reference set of mass spectrometry data obtained from blood-based samples obtained from a multitude of early stage non-small-cell cancer patients used in classifier development. The mass spectrometry data includes feature values for features listed in Appendix A.

In another aspect, a programmed computer is described configured for making a prediction of the risk of recurrence of cancer in an early stage non-small-cell lung cancer patient. The programmed computer includes a processing unit and a memory storing code and classifier parameters such that the computer is configured as a hierarchical classifier as per FIG. 3 or FIG. 14. The memory further storing a reference set of mass spectral data from a multitude of early stage non-small cell lung cancer patients including feature values of the features listed in Appendix A.

In another aspect, a method for detecting a class label in an early stage non-small-cell lung cancer patient is disclosed. The method includes steps of (a) conducting mass spectrometry on a blood-based sample obtained from the patient and obtaining integrated intensity values in the mass spectral data of a multitude of pre-determined mass-spectral features, and (b) operating on the mass spectral data with a programmed computer implementing a classifier, wherein the programmed computer performs a hierarchical classification procedure on the mass spectrometry data, including a first classifier (Classifier A) producing a class label in the form of high risk or low risk or the equivalent, and if the Classifier A produces the high risk label the sample is classified by a second classifier (Classifier B) generating a classification label of highest risk or high/intermediate risk or the equivalent. In the operating step the classifier compares the integrated intensity values obtained in step (a) with feature values of a reference set of class-labeled mass spectral data obtained from blood-based samples obtained from a multitude of other early stage non-small-cell lung cancer patients with a classification algorithm and detects a class label for the sample in accordance with the hierarchical classification schema.

In another aspect, a method is described for performing a risk assessment of recurrence of cancer in an early stage non-small-cell lung cancer patient having surgery to treat the cancer. The method includes steps of: (1) obtaining a pre-surgery blood-based sample from the patient, performing mass spectrometry on the sample and obtaining the integrated intensity values of the features listed in Appendix A, and then classifying the mass spectrum of the sample with a computer-based classifier developed from a set of blood-based samples obtained from other early stage NSCLC patients, the classifier producing a label of high or highest risk of recurrence or the equivalent and low or lowest risk of recurrence or the equivalent; (2) if the sample is not classified as high or highest risk of recurrence in accordance with the classification produced in step (1), obtaining a further blood-based sample from the patient after the surgery and conducting mass spectrometry on the blood-based sample including obtaining integrated intensity values of the features listed in Appendix A; and (3) classifying the mass spectrum of the sample obtained in (2) in accordance with a computer-based classifier developed from a set of blood-based samples obtained from other early stage NSCLC patients after surgery, wherein the classifier of this paragraph (3) generates a class label of either G1 or the equivalent or G2 or the equivalent, with G2 class label associated with a prediction that the patient will have a lower risk of recurrence as compared to risk of recurrence associated with the class label G1.

### BRIEF DESCRIPTION OF THE DRAWINGS

FIG. 1A is a plot of time-to-recurrence (TTR) and FIG. 1B is a plot of overall survival (OS) for the classifier development cohort.

FIG. 2 is a flow-chart showing a deep learning classifier development procedure we used to develop classifiers A, B and C described in detail below.

FIG. 3 is a hierarchical schema showing the combination of classifiers A, B and C to generate a class label for a blood-based sample from an early stage NSCLC patient; the class label is a prediction of the risk of recurrence of the cancer following surgery. FIG. 3 is implemented in program code of a computer which applies the classifiers A, B and C to the mass spectral data of a blood-based sample of the NSCLC patient, for example in a testing laboratory.

FIG. 4A and FIG. 4B are plots of time-to-event outcomes by binary test classification produced by Classifier A on the development set. FIG. 4A shows TTR and FIG. 4B shows OS.

FIGS. 5A and 5B are plots of time-to-event outcomes of the high risk group stratified into highest and high/int risk, produced by Classifier B. FIG. 5A shows TTR and FIG. 5B shows OS.

FIG. 6 is a plot of time-to-event outcomes of the low risk group stratified from ST100 spectra into lowest and low/int risk, produced by Classifier C.

FIG. 7 is a plot of time-to-event outcomes of the low risk group stratified from ST1 spectra into lowest and low/int risk produced by Classifier C.

FIGS. 8A and 8B are plots of time-to-event outcomes by 4-way test classifications (lowest, low/int. high/int, and highest) produced by the combination of Classifiers A, B and C per FIG. 3, FIG. 8A shows OS and FIG. 8B shows TTR, Both plots show four curves; in FIG. 8A there are no events in either the low/int or the lowest risk group, so the two curves are both horizontal lines that lie on top of each other.

FIG. 9A is a plot of RFS (recurrence free survival) and FIG. 9B is a plot of OS (overall survival) for the classifier redevelopment cohort described in Section 7 of the Detailed Description.

FIGS. 10A and 10B are plots of time to event outcomes by binary test classification produced by Classifier A in the redevelopment exercise of Section 7; FIG. 10A is a plot of RFS and FIG. 10B is a plot of OS.

FIGS. 11A and 11B are plots of time to event outcomes by binary test classification produced by Classifier B in the redevelopment exercise of Section 7; FIG. 11A is a plot of RFS and FIG. 11B is a plot of OS.

FIGS. 12A and 12B are plots of time to event outcomes by binary test classification produced by Classifier C in the redevelopment exercise of Section 7; FIG. 12A is a plot of RFS and FIG. 12B is a plot of OS.

FIGS. 13A and 13B are plots of time to event outcomes by a four-way hierarchical test classification schema using FIG. 3 in the redevelopment exercise of Section 7; FIG. 13A is a plot of RFS and FIG. 13B is a plot of OS.

FIG. 14 is a hierarchical schema showing the combination of classifiers A, B and C to generate a class label for a blood-based sample from an early stage NSCLC patient as an alternative to the schema of FIG. 3. The class label is a prediction of the risk of recurrence of the cancer following surgery. FIG. 14 is implemented in program code of a computer which applies the classifiers A, B and C to the mass spectral data of a blood-based sample of the NSCLC patient, for example in a testing laboratory.

FIGS. 15A and 15B are plots of time-to-event outcomes by 3-way test classifications (lowest, intermediate and highest) produced by the combination of Classifiers A, B and C per FIG. 14 in the redevelopment exercise of Section 7. FIG. 15A shows RFS and FIG. 15B shows OS,

FIGS. 16A and 16B are plots of the time to event outcomes produced by the post-surgery classifier of Section 8, in addition to the time to event data for the highest risk of recurrence patients from the classifier of Section 7. FIG. 16A shows RFS and FIG. 16B shows OS.

FIGS. 17A and 17B are plots of time to event outcomes split on both pre-surgery classification (Int./Lowest, labels produced by the pre-surgery classifier of Section 7) as well as post-surgery classification (G1/G2), produced by the post-surgery classifier of Section 8) for samples not classified as highest-risk by the pre-surgery classifier of Section 7. FIG. 17A shows RFS and FIG. 17B shows OS.

### DETAILED DESCRIPTION

Overview

This document will describe the development of a blood-based test and related machine-implemented classifier which makes a prediction of whether a blood sample for an early stage NSCLC patient indicates that the patient is at high risk of recurrence of the cancer. The classifier is developed from mass spectral data obtained from serum samples from a multitude of early stage NSCLC patients. Once the classifier is developed, as explained in this document, it is used to generate a class label for mass spectral data of a blood sample for an early stage NSCLC patient indicating, i.e., predicting, whether the patient providing that blood sample is at high risk of recurrence of the cancer after surgery. The blood sample can be obtained prior to, at the time of, or after surgery to remove the cancer.

Section 1 provides a description of a set of serum samples obtained from early stage (IA or IB) NSCLC patients which were used to develop the test of this disclosure.

Section 2 explains our methods of obtaining mass spectral data from the serum samples. The methods of Section 2 make use of mass spectral data acquisition and processing steps which are described extensively in the prior patent applications and issued patents of the Assignee Biodesix, Inc. Reference is made to such patents and applications for further details.

Section 3 describes a deep learning classifier development method we used to generate a classifier from the mass spectral data in a classifier development set, which is known as the “Diagnostic Cortex” method of the Assignee and described in previous patent literature. The methodology was performed on the mass spectral data obtained as explained in Section 2 and makes use of mass spectral feature definitions (m/z ranges) in the data which are described in Appendix A.

Section 4 describes a hierarchical combination of classifiers that are used to classify a blood-based sample as either high risk of cancer recurrence, intermediate risk, or low risk. A first classifier (“Classifier A” in the following discussion) was developed which is a binary classifier which splits the development sample set as either High Risk or Low Risk. A practical test could be implemented using just Classifier A. A second classifier (“Classifier B”) stratifies the high risk group defined by the first classifier into two groups with highest (“highest”) and intermediate (“high/int”) risk of recurrence. In a practical testing environment, in one possible implementation, the blood sample is subject to mass spectrometry and if the Classifier A returns a High Risk classification label, it is subject to classification by Classifier B and if Classifier B returns a Highest Risk label (or the equivalent) the patient is predicted to have a high risk of recurrence and is guided towards more aggressive treatment. If the sample is classified by Low Risk by Classifier A, or as “high/int” risk by Classifier B, the patient is not guided towards the more aggressive treatment. However, intermediate or low risk classification labels may still be used to guide treatment or plan surgery on the cancer.

An optional third classifier is described (“Classifier C”) which stratifies the low risk group defined by the first classifier into two groups with lowest (“lowest”) and intermediate (“low/int”) risk of recurrence.

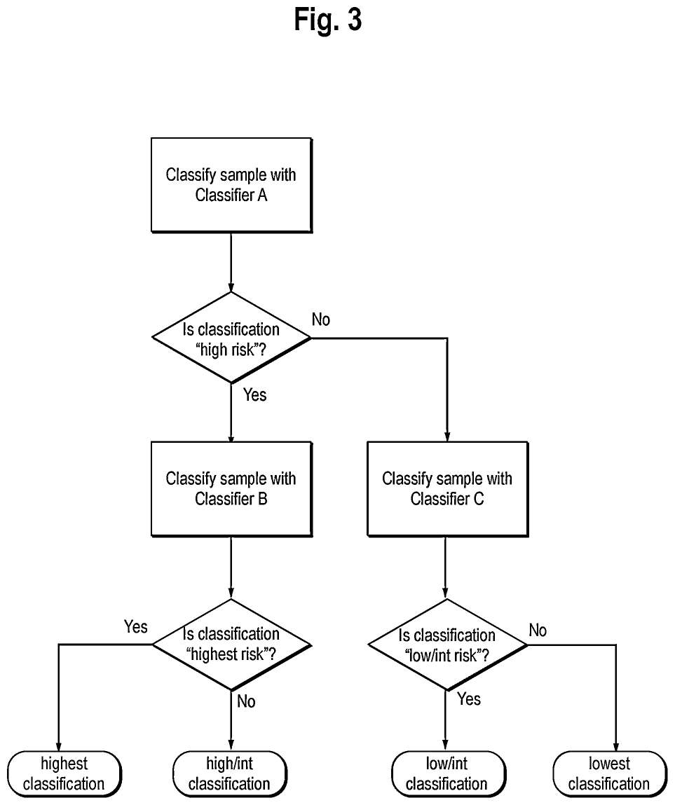

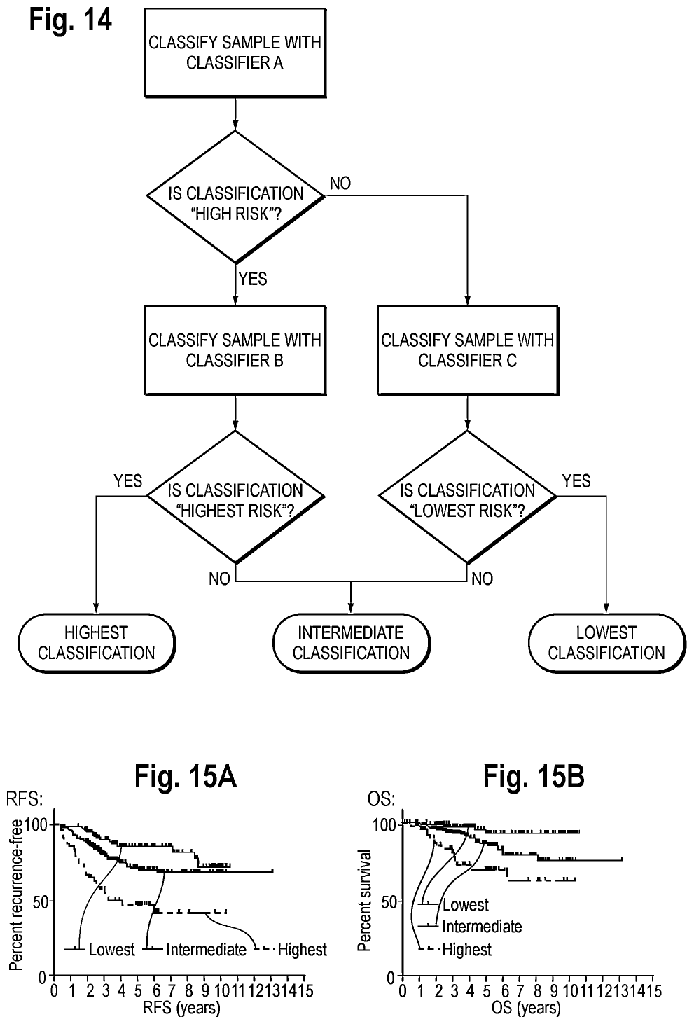

In one possible embodiment a practical test employs the hierarchical combination of all three classifiers using program logic in accordance with FIG. 3 or FIG. 14. Alternatively, a test for identifying high risk of recurrence patients can be implemented using just Classifiers A and B, or just Classifier A, or Classifiers A, B and C.

In Section 4 we also show that the stratification produced by classifiers A, B and C remained significant in multivariate analysis including histology, tumor size, gender and age. This indicates that the stratification is providing information that is additional and complementary to these clinicopathological factors,

Section 5 describes our work associating the test classifications with biological processes using a method known as protein set enrichment analysis (PSEA). Using multivariate techniques we defined specific states of the host biology related phenotypes associated with risk of recurrence from pre-surgery measurements of the circulating proteome, Biology underlying these disease states was investigated. Patients in the highest risk classification group had significantly elevated levels of acute phase response, acute inflammatory response, wound healing and complement. Data indicate that systemic host effects related to the circulating proteome measurable from pre-surgery samples may play an important role in assessing risk of recurrence in early stage NSCLC independent of type of recurrence, including new primaries. The associated biological processes have previously been shown to be related to immune checkpoint resistance in metastatic melanoma and lung cancer, and may be related to a particular state of the host's immune system.

Section 6 describes a practical laboratory testing environment in which the methods of this disclosure can be practiced.

Section 7 describes a redevelopment of the test described in Sections 1-6 but using additional samples from a validation set that we had available. Our work described in this section envisions a ternary or three-way classification schema (see FIG. 14) by which an early stage NSCLC patient can be classified as having a high, intermediate, or low risk of recurrence of the cancer. This ternary classification schema also uses classifiers A, B and C, as described in previous sections, although their performance characteristics (evidenced by Kaplan-Meier Plots) differ slightly due to the larger sample set used for redevelopment of the classifiers in this Section.

Section 8 describes a classifier developed from samples obtained from NSCLC patients post-surgery. This classifier stratifies patients into a group with higher risk of recurrence or lower risk. The classifier of Section 8 could be used in conjunction with the classifier (or combination of classifiers) described in Sections 4 or 7.

Section 9, Further Considerations, describes additional details on how practical tests in accordance with this disclosure can be implemented in practice.

Section 1: Classifier Development Sample Set

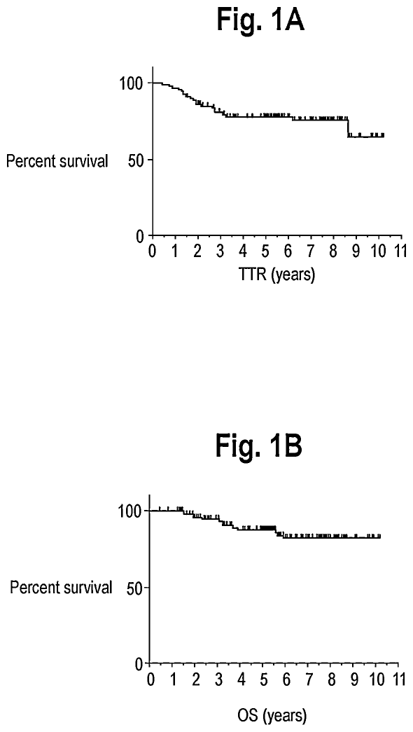

Serum samples taken either at or pre-surgery were available from 124 patients with Stage IA or IB NSCLC. No patients received adjuvant therapy following surgery. Median follow up of these patients was 5.1 years (median (range) for patients alive: 4,9 (0.5-10.1) years). Patient characteristics are summarized in Table 1. FIGS. 1A and 1B show the time-to-recurrence (TTR) and overall survival (OS) for the cohort. Recurrence was identified in 27 patients (22%). Death was observed for 17 patients (14%); however, date of death was unknown for 3 of these patients, who were therefore censored for survival at last follow up date.

TABLE 1
Patient characteristics for the development cohort

| | | n(%) | |
|---|---|---|---|
| Gender | Female | 78 | (63) |
| | Male | 46 | (37) |
| Race | White (including Hispanic) | 110 | (89) |
| | Other | 13 | (10) |
| | NA | 1 | (1) |
| Histology | adenocarcinoma | 107 | (86) |
| | other | 17 | (14) |
| Smoking | Current | 7 | (6) |
| Status | Packyears median (range) | 65 | (20-128) |
| | Former | 54 | (44) |
| | Packyears median (range) | 40 | (5-180) |
| | Never | 13 | (10) |
| | NA* | 50 | (40) |
| TNM T | 1 | 102 | (82) |
| | 1a | 51 | |
| | 1b | 19 | |
| | Not specified | 32 | |
| | 2 | 19 | (15) |
| | 2a | 12 | |
| | 2b | 0 | |
| | Not specified | 7 | |
| | 3 | 3 | (2) |
| Pleural | yes | 9 | (7) |
| Invasion | no | 115 | (93) |
| LVI | yes | 7 | (6) |
| | no | 117 | (94) |
| # nodules | 1 | 120 | (97) |
| | Size median, range | 1.8 | (0.5-13) |
| | 2 | 4 | (3) |
| | Total Size (median, range) | 4.0 | (2.9-4.5) |
| Age | Median (range) | 71 | (46-89) |

*mainly former or current (based on pack-years

Eleven of the 27 patients recurring died while under follow up: 10 from lung cancer, and the remaining 1 from unspecified causes.

Of the 27 recurrences, 6 (22%) were distant, 11 (41%) were locoregional, and 10 (37%) were new primaries. Four recurrences were observed within 1 year (2 new primary, 2 locoregional), a further 13 were observed between 1 and 2 years after surgery (3 distant, 6 locoregional, and 4 new primaries).

Section 2: Mass Spectral Data Acquisition and Processing

The serum samples explained in Section 1 were subject to mass spectrometry as explained in this section. Once the classifiers were developed and fully defined, the feature values of features listed in Appendix A were then saved as a reference set in computer memory for use in conducting a classification procedure on a new (previously unseen) sample, for example at the time of use to make a prediction as to a given early stage NSCLC patient.

Sample Preparation

Samples were thawed and 3 μl aliquots of each test sample and quality control serum (a pooled sample obtained from serum of thirteen healthy patients, purchased from

Conversant Bio, “SerumP4”) spotted onto VeriStrat serum cards (Therapak). The cards were allowed to dry for 1 hour at ambient temperature after which the whole serum spot was punched out with a 6 mm skin biopsy punch (Acuderm). Each punch was placed in a centrifugal filter with 0.45 μm nylon membrane (VWR). One hundred pi of HPLC grade water (JT Baker) was added to the centrifugal filter containing the punch. The punches were vortexed gently for 10 minutes then spun down at 14,000 rcf for two minutes. The flow-through was removed and transferred back on to the punch for a second round of extraction. For the second round of extraction, the punches were vortexed gently for three minutes then spun down at 14,000 rcf for two minutes. Twenty microliters of the filtrate from each sample was then transferred to a 0.5 ml eppendorf tube for MALDI analysis.

All subsequent sample preparation steps were carried out in a custom designed humidity and temperature control chamber (Coy Laboratory). The temperature was set to 30 ° C. and the relative humidity at 10%.

An equal volume of freshly prepared matrix (25 mg of sinapinic acid per 1 ml of 50% acetonitrile:50% water plus 0.1% TFA) was added to each 20 μl serum extract and the mix vortexed for 30 sec. The first three aliquots (3×2 μl) of sample:matrix mix were discarded into the tube cap. Eight aliquots of 2 μl sample:matrix mix were then spotted onto a stainless steel MALDI target plate (SimulTOF). The MALDI target was allowed to dry in the chamber before placement in the MALDI mass spectrometer.

QC samples (SerumP4) were added to the beginning (two preparations) and end (two preparations) of each batch run.

Spectral Acquisition

MALDI spectra were obtained using a MALDI-TOF mass spectrometer (SimulTOF 100, s/n: LinearBipolar 11.1024.01 or SimulTOF One, sin ClinicalAnalyzer 15.1032.01: from SimulTOF Systems, Marlborough, Mass., USA). The instruments were operated in positive ion mode, with ions generated using a 349 nm, diode-pumped, frequency-tripled Nd:YLF laser firing at a laser repetition rate of 0.5 kHz (SimulTOF100) or 1 kHz (SimulTOF One). External calibration was performed using the following peaks in the QC serum spectra: m/z=3320, 4158,7338, 6636.7971, 9429,302, 13890.4398, 15877,5801 and 28093.951.

Spectra from each MALDI spot were collected as 800 shot spectra that were ‘hardware averaged’ as the laser fires continuously across the spot while the stage is moving at a speed of 0.25 mm/sec (SimulTOF 100) or 0.5 mm/sec (SimulTOF One), A minimum intensity threshold of 0.01 V or 0.003 V for the SimulTOF 100 and SimulTOF One, respectively was used to discard any ‘flat line’ spectra. All 800 shot spectra with intensity above this threshold were acquired without any further processing.

The spectral acquisition made use of the techniques described in the Biodesix U.S. Pat. No. 9,279,798, a technique which is referred to as “Deep MALDI” in this document.

Spectral Processing

Each raster spectrum of 800 shots was processed through an alignment workflow to align prominent peaks to a set of 43 alignment points (see table 2). A filter was applied that essentially smooths noise and the spectra were background subtracted for peak identification. Once peaks had been identified, the filtered spectra (without background subtraction) were aligned. Additional filtering parameters required that raster spectra have at least 20 peaks and used at least 5 alignment points to be included in the pool of rasters used to assemble the average spectrum.

TABLE 2
Alignment points
used to align the
raster spectra
m/z

3168.00
4153.48
4183.00
4792.00
5773.00
5802.00
6432.79
6631.06
7202.00
7563.00
7614.00
7934.00
8034.00
8206.35
8684.25
8812.00
8919.00
8994.00
9133.25
9310.00
9427.00
10739.00
10938.00
11527.06
12173.00
12572.38
12864.24
13555.00
13762.87
13881.55
14039.60
14405.00
15127.49
15263.00
15869.06
17253.06
18629.76
21065.65
23024.00
28090.00
28298.00

Averages were created from the pool of aligned and filtered raster spectra. A random selection of 500 raster spectra was averaged to create a final analysis spectrum for each sample of 400000 shots.

Although the m/z range is collected from 3-75 KDa, the range for spectral processing is limited to 3-30 KDa including feature generation, as features above 30 KDa have poor resolution and were not found to be reproducible at a feature value level.

We performed background estimation and subtraction, and normalization of the spectra, including a partial ion current normalization, the details of which are not particularly important. We also performed an average spectra alignment to address minor differences in peak positions in the spectra by defining a set of calibration points (m/z positions) used to align spectral averages. We defined a set of 282 features (see Appendix A) that had been discovered and well established from our previous Deep MALDI spectral analysis work relating to blood-based samples in cancer patients.

We further performed a batch correction step making use of quality control reference sample spectra similar to the methodology described in our prior U.S. Pat. No. 9,279,798, the details of which are not particularly important. Following batch correction, a final partial ion current by feature normalization step was applied to the feature tables to account for changes related to m/z dependent corrections, similar to the method described in U.S. Pat. No. 10,007,766, the details of which are not particularly important. The normalization scalars used for partial ion current normalization were not found to be associated with the time to recurrence groups.

In a final step, a trim or pruning of the feature list of Appendix A was done. In particular, eight features of Appendix A were included in the preprocessing that are ill-suited for inclusion in new classifier development in this situation as they are related to hemolysis. It has been observed that these large peaks are useful for stable batch corrections because once in the serum, they appear stable over time and resistant to modifications. However, these peaks are related to the amount of red blood cell shearing during the blood collection procedure and should not be used for test development beyond feature table corrections in preprocessing. The features listed in Appendix A marked with an asterisk (*) were removed from the final feature table, yielding a total of 274 features used for classifier development.

Section 3: Classifier Development Method (Diagnostic Cortex)

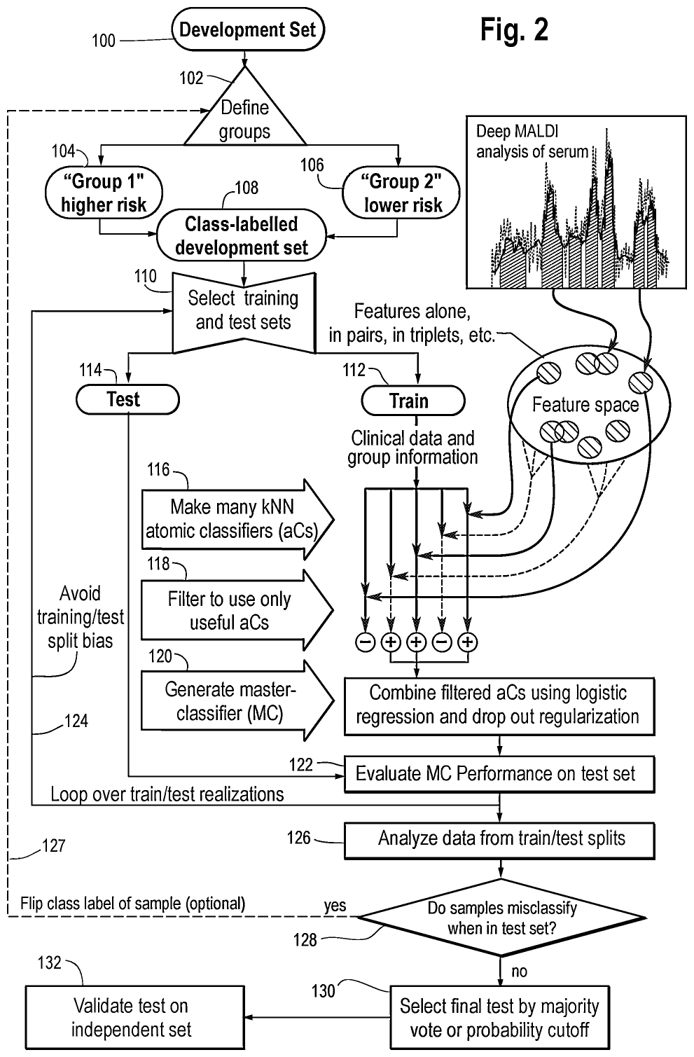

The new classifier development process was carried out using the “Diagnostic Cortex”® procedure shown in FIG. 2. This procedure, implemented in a general purpose computer system, is described at length in the patent literature, see U.S. Pat. No. 9,477,906. See also FIGS. 8A-8B and the corresponding discussion of U.S. Pat. No. 10,007,766. An overview of the process will be described and then the specifics and results for the three classifiers developed and classification results will be described later on.

This document describes three different classifiers, Classifier A, Classifier B, and Classifier C which are used in a hierarchical manner to generate a class label to indicate the risk of recurrence of a patient blood sample. See FIGS. 3 and 14 for configurations of the hierarchical structure of the classifiers. The procedure of FIG. 2 was repeated three times in order to generate the three classifiers (A, B and C), and in each iteration of the procedure of FIG. 2 certain details as to the parameters for the procedure of FIG. 2 differed so as result in three different classifiers, as will be explained below.

Since the generation of classifiers A, B and C each used the methodology of FIG. 2 some explanation of the method will be provided at a high level. The interested reader is referred to U.S. Pat. Nos. 9,477,906 and 10,007,766 for other examples and further explanations as to how the procedure works.

In contrast to standard applications of machine learning focusing on developing classifiers when large training data sets are available, the big data challenge, in bio-life-sciences the problem setting is different. Here we have the problem that the number (n) of available samples, arising typically from clinical studies, is often limited, and the number of attributes (measurements) (p) per sample usually exceeds the number of samples. Rather than obtaining information from many instances, in these deep data problems one attempts to gain information from a deep description of individual instances. The present methods of FIG. 2 take advantage of this insight, and are particularly useful, as here, in problems where p»n.

The method includes a first step of obtaining measurement data for classification from a multitude of samples, i.e., measurement data reflecting some physical property or characteristic of the samples. The data for each of the samples consists of a multitude of feature values, and a class label. In this example, the data takes the form of mass spectrometry data, in the form of feature values (integrated peak intensity values at a multitude of m/z ranges or peaks, see Appendix A). This is indicated by “development set” 100 in FIG. 2. This step is explained at length above in Section 2, and is obtained for set of patient blood-based samples which were used to generate the classifiers, see Section 1.

At step 102, a label associated with some attribute of the sample is assigned (for example, patient high risk or low risk of recurrence, “Group1”, “Group2” etc. the precise moniker of the label is not important). In this example, the class labels were assigned by a human operator to each of the samples after investigation of the clinical data associated with the sample. In this example, the sample set is split into two groups, “Group1” (104) being the label assigned to patients at a relatively high risk of recurrence and “Group2” (106) being the label assigned to patients with a relatively lower risk of recurrence, based on the clinical data associated with the samples. This results in a class-labelled development set shown at 108.

Then, at step 110, the class-labeled development sample set 108 is split into a training set 112 and a test set 114. The training set is used in the following steps 116, 118 and 120.

In the training step, the process continues with a step 116 of constructing a multitude of individual mini-Classifiers using sets of feature values from the samples up to a pre-selected feature set size s (s=integer 1 . . . p). For example a multiple of individual mini- (or “atomic”) Classifiers could be constructed using a single feature (s=1), or pairs of features (s=2), or three of the features (s=3), or even higher order combinations containing more than 3 features. The selection of a value of s will normally be small enough to allow the code implementing the method to run in a reasonable amount of time, but could be larger in some circumstances or where longer code run-times are acceptable. The selection of a value of s also may be dictated by the number of measured variables (p) in the data set, and where p is in the hundreds, thousands or even tens of thousands, s will typically be 1, or 2 or possibly 3, depending on the computing resources available. In the present work, s took a value of 1, 2 or 3 as explained later. The mini-Classifiers of step 116 execute a supervised learning classification algorithm, such as k-nearest neighbors (kNN), in which the values for a feature, pairs or triplets of features of a sample instance are compared to the values of the same feature or features in a training set and the nearest neighbors (e.g., k=9) in an s-dimensional feature space are identified and by majority vote a class label is assigned to the sample instance for each mini-Classifier. In practice, there may be thousands of such mini-Classifiers depending on the number of features which are used for classification.

The method continues with a filtering step 118, namely testing the performance, for example the accuracy, of each of the individual mini-Classifiers to correctly classify the sample, or measuring the individual mini-Classifier performance by some other metric (e.g. the Hazard Ratios (HRs) obtained between groups defined by the classifications of the individual mini-Classifier for the training set samples) and retaining only those mini-Classifiers whose classification accuracy, predictive power, or other performance metric, exceeds a pre-defined threshold to arrive at a filtered (pruned) set of mini-Classifiers. The class label resulting from the classification operation may be compared with the class label for the sample known in advance if the chosen performance metric for mini-Classifier filtering is classification accuracy. However, other performance metrics may be used and evaluated using the class labels resulting from the classification operation. Only those mini-Classifiers that perform reasonably well under the chosen performance metric for classification are maintained in the filtering step 118. Alternative supervised classification algorithms could be used, such as linear discriminants, decision trees, probabilistic classification methods, margin-based Classifiers like support vector machines, and any other classification method that trains a Classifier from a set of labeled training data,

To overcome the problem of being biased by some univariate feature selection method depending on subset bias, we take a large proportion of all possible features as candidates for mini-Classifiers. We then construct all possible kNN classifiers using feature sets up to a pre-selected size (parameter s). This gives us many “mini-Classifiers”: e.g. if we start with 100 features for each sample (p=100), we would get 4950 “mini-Classifiers” from all different possible combinations of pairs of these features (s=2), 161,700 mini-Classifiers using all possible combination of three features (s=3), and so forth. Other methods of exploring the space of possible mini-Classifiers and features defining them are of course possible and could be used in place of this hierarchical approach. Of course, many of these “mini-Classifiers” will have poor performance, and hence in the filtering step c) we only use those “mini-Classifiers” that pass predefined criteria. These filtering criteria are chosen dependent on the particular problem: If one has a two-class classification problem, one would select only those mini-Classifiers whose classification accuracy exceeds a pre-defined threshold, i.e., are predictive to some reasonable degree, Even with this filtering of “mini-Classifiers” we end up with many thousands of “mini-Classifier” candidates with performance spanning the whole range from borderline to decent to excellent performance.

The method continues with step 120 of generating a Master Classifier (MC) by combining the filtered mini-Classifiers using a regularized combination method. In one embodiment, this regularized combination method takes the form of repeatedly conducting a logistic training of the filtered set of mini-Classifiers to the class labels for the samples. This is done by randomly selecting a small fraction of the filtered mini-Classifiers as a result of carrying out an extreme dropout from the filtered set of mini-Classifiers (a technique referred to as drop-out regularization herein), and conducting logistic training on such selected mini-Classifiers. While similar in spirit to standard classifier combination methods (see e.g. S. Tulyakov et al., Review of Classifier Combination Methods, Studies in Computational Intelligence, Volume 90, 2008, pp. 361-386), we have the particular problem that some “mini-Classifiers” could be artificially perfect just by random chance, and hence would dominate the combinations. To avoid this overfitting to particular dominating “mini-Classifiers”, we generate many logistic training steps by randomly selecting only a small fraction of the “mini-Classifiers” for each of these logistic training steps. This is a regularization of the problem in the spirit of dropout as used in deep learning theory. In this case, where we have many mini-Classifiers and a small training set we use extreme dropout, where in excess of 99% of filtered mini-Classifiers are dropped out in each iteration.

In more detail, the result of each mini-Classifier is one of two values, either “Groupl” or “Group2” in this example. We can then combine the results of the mini-Classifiers by defining the probability of obtaining a “Group1” label via standard logistic regression (see e.g. http://en.wikipedia.org/wiki/Logistic_regression)

$$P ( “ Group 1 ” \vert feature values for a spectrum ) = \frac{\begin{matrix}exp ( \sum {w}_{mc} / ( mc ( feature values ) ) ) \\ mini Classifiers\end{matrix}}{Normalization} \qquad Eq . ( 1 )$$

where l(mc(feature values))=1, if the mini-Classifier mc applied to the feature values of a sample returns “Group2”, and 0 if the mini-Classifier returns “Group1”. The weights w_{mc} for the mini-Classifiers are unknown and need to be determined from a regression fit of the above formula for all samples in the training set using +1 for the left hand side of the formula for the Group2-labeled samples in the training set, and 0 for the Group1-labeled samples, respectively. As we have many more mini-Classifiers, and therefore weights, than samples, typically thousands of mini-Classifiers and only tens of samples, such a fit will always lead to nearly perfect classification, and can easily be dominated by a mini-Classifier that, possibly by random chance, fits the particular problem very well. We do not want our final test to be dominated by a single special mini-Classifier which only performs well on this particular set and is unable to generalize well. Hence we designed a method to regularize such behavior: Instead of one overall regression to fit all the weights for all mini-Classifiers to the training data at the same time, we use only a few of the mini-Classifiers for a regression, but repeat this process many times in generating the master classifier. For example we randomly pick three of the mini-Classifiers, perform a regression for their three weights, pick another set of three mini-Classifiers, and determine their weights, and repeat this process many times, generating many random picks, i.e. realizations of three mini-Classifiers. The final weights defining the master Classifier are then the averages of the weights over all such realizations. The number of realizations should be large enough that each mini-Classifier is very likely to be picked at least once during the entire process. This approach is similar in spirit to “drop-out” regularization, a method used in the deep learning community to add noise to neural network training to avoid being trapped in local minima of the objective function.

In a variation of the above method, which was used in the present classifier generation exercises, we saved all of the weights w_{mc} for each dropout iteration and average the P from Eq. 1 calculated for a sample over all the dropout iterations (instead of averaging the weights for the mCs over the dropout iterations and only storing those and then working out the result for a new sample from the averaged weights). We have some description of this difference in U.S. Provisional patent application Ser. No. 62/649,762 filed Mar. 29, 2018, where some of the classifiers use the original weight averaging method and others use the new probability averaging method. The interested reader is directed to that description, which is incorporated by reference herein. The probability averaging technique has some technical advantages when the regression does not converge (“separable” cases for a dropout iteration) or converges slowly, as the probabilities can converge (or can converge faster) even though the weights do not (or converge slowly).

Other methods for performing the regularized combination method in step 120 that could be used include:
Logistic regression with a penalty function like ridge regression (based on Tikhonov regularization, Tikhonov, Andrey Nikolayevich (1943). “ [symbol] [symbol] [symbol] [symbol] ” [On the stability of inverse problems]. Doklady Akademii Nauk SSSR 39 (5): 195-198.)
The Lasso method (Tibshirani, R. (1996), Regression shrinkage and selection via the lasso. J. Royal. Statist. Soc B., Vol. 58, No. 1, pages 267-288).
Neural networks regularized by drop-out (Nitish Shrivastava, “Improving Neural Networks with Dropout”, Master's Thesis, Graduate Department of Computer Science, University of Toronto), available from the website of the University of Toronto Computer Science department.

General regularized neural networks (Girosi F. et al, Neural Computation, (7), 219 (1995)).

The above-cited publications are incorporated by reference herein. Our approach of using drop-out regularization has shown promise in avoiding over-fitting, and increasing the likelihood of generating generalizable tests, i.e, tests that can be validated in independent sample sets.

“Regularization” is a term known in the art of machine learning and statistics which generally refers to the addition of supplementary information or constraints to an underdetermined system to allow selection of one of the multiplicity of possible solutions of the underdetermined system as the unique solution of an extended system. Depending on the nature of the additional information or constraint applied to “regularize” the problem (i.e. specify which one or subset of the many possible solutions of the unregularized problem should be taken), such methods can be used to select solutions with particular desired properties (e,g. those using fewest input parameters or features) or, in the present context of classifier training from a development sample set, to help avoid overfitting and associated lack of generalization (i.e., selection of a particular solution to a problem that performs very well on training data but only performs very poorly or not all on other datasets), See e.g., https://en.wikipedia.org/wiki/Regularization (mathematics). One example is repeatedly conducting extreme dropout of the filtered mini-Classifiers with logistic regression training to classification group labels. However, as noted above, other regularization methods are considered equivalent. Indeed it has been shown analytically that dropout regularization of logistic regression training can be cast, at least approximately, as L2 (Tikhonov) regularization with a complex, sample set dependent regularization strength parameter λ. (S Wager, S Wang, and P Liang, Dropout Training as Adaptive Regularization. Advances in Neural Information Processing Systems 25, pages 351-359, 2013 and D Helmbold and P Long, On the Inductive Bias of Dropout, JMLR, 16:3403-3454, 2015). In the term “regularized combination method” the “combination” simply refers to the fact that the regularization is performed over combinations of the mini-Classifiers which pass filtering. Hence, the term “regularized combination method” is used to mean a regularization technique applied to combinations of the filtered set of mini-Classifiers so as to avoid overfitting and domination by a particular mini-Classifier.

Still referring to FIG. 2, at step 122 the performance of the master classifier generated at step 120 is then evaluated by how well it classifies the subset of samples forming the test set.
As indicated by the loop 124, steps 110, 116, 118, 120 and 122 are repeated in the programmed computer for different realizations of the separation of the set of samples into test and training sets (at step 110), thereby generating a plurality of master classifiers, one for each realization of the separation of the set of samples into training and test sets or iteration through loop 124.

The performance of the master classifier is evaluated for all the realizations of the separation of the development set of samples into training and test sets in step 126. If there are some samples which persistently misclassify when in the test set, as indicated by the block 128 the process optionally loops back as indicated at loop 127 and steps 102, 110, 116, 118, and 120 are repeated with flipped class labels for such misclassified samples.

The method continues with step 130 of defining a final classifier from one or a combination of more than one of the plurality of master classifiers. In the present example, the final classifier is defined as a majority vote or ensemble average of all the master classifiers resulting from each separation of the sample set into training and test sets, or alternatively by an average probability cutoff, selecting one Master Classifier that has typical performance, or some other procedure. At step 132, the classifier (or test) developed from the procedure of FIG. 2 and defined at step 130 is validated on an independent sample set.

Section 4: Hierarchical Combination of Classifiers

As explained previously, the methodology of FIG. 2 was performed several times to develop different Classifiers, and in particular a first classifier (Classifier A), a second classifier (Classifier B), and a third classifier (classifier C). In one possible implementation, these three classifiers are combined in a hierarchical manner to develop a label for a patient sample indicating risk of recurrence using logical operations on the output of the three classifiers, see the hierarchical schema shown in FIG. 3 or FIG. 14. In this section we explain the splits or separations in the development sets produced by the different classifiers as an exercise in classifier development. As a test on a new, previously unseen sample, the sample is subject to the classifiers as explained in the schema of FIG. 3 or 14.

A. Classifier A—First Split of the Sample Set.

A first split of the sample set was achieved using a classifier developed in accordance with FIG. 2 and the above detailed description, referred to as Classifier A. This classifier split the development set into “high” risk of recurrence (Groupl label) and “low” risk of recurrence (Group2 label) groups. Performance data for Classifier A will be discussed in detail below. Classifier A was developed with the following parameters and design (making reference to FIG. 2):

A “label-flip” approach was used (loop 127), in which the training class labels (at step 102), and master classifiers (resulting from step 120) were simultaneously iteratively refined.
The training class labels for initiation of the iterative refinement were obtained from a previous classifier that used feature deselection and had been trained without label flip for patients recurring versus patients with no recurrence.
The atomic classifiers (step 116) were k=9 k-nearest neighbor classifiers
Atomic classifiers used 1, 2, or 3 mass spectral features (parameter s)
Feature deselection was used, with approximately 170 features discarded (100 used) at each step of the iterative refinement process. Feature deselection methods are explained in the prior patent literature, see e.g. U.S. patent application publication 201610321561, the content of which is incorporated by reference herein.
mini-classifier filtering (step 118) was by time-to-recurrence (TTR) hazard ratio, with limits 2.8-10 for flip 0, 2.5-10 for flip 1 and 2.4-10 for flip 2. (Flip 0, 1 and 2 representing three iterations through loop 127 in FIG. 2).
500,000 dropout iterations were used in step 120, each iteration retaining 10 atomic or mini-Classifiers.
Master classifiers resulting from 625 test/training splits (step 110) were ensemble averaged to generate the final test at step 130.

B. Classifier B: Second split of he high risk outcome group from the first split (Classifier A)

The first split of the sample set from Classifier A resulted in a high risk or “poor” outcome group of 56 patients, with 20 recurrers. To further stratify by outcome, the samples in this high risk or “poor” outcome group were split with a second classifier, “Classifier B” developed in accordance with FIG. 2. This Classifier B was developed using the following parameters and design (again with reference to FIG. 2):
A “label-flip” approach was used, in which training class labels and classifier were simultaneously iteratively refined.
The training class labels for initiation of the iterative refinement were defined so that the patients with lowest TTR times (regardless of event or no event) were in one group and the patients with highest TTR times were in the other group.
The atomic classifiers were k=9 k-nearest neighbor classifiers
Atomic classifiers used 1 or 2 mass spectral features.
No feature deselection was used. All 274 features and their pairs were considered in the atomic classifier filtering step.
Filtering was by TTR hazard ratio, with limits 2.5-10.
150,000 dropout iterations were used, each retaining 10 atomic classifiers.
The master classifiers resulting from 625 test/training splits were ensemble averaged to arrive at the final classifier definition at step 130.

C. Classifier C: Second split of the low risk outcome group from the first split (Classifier A)

The first split of the sample set performed by Classifier A resulted in a “good” or low risk outcome group of 68 patients, with 7 recurrers. To further stratify by outcome, this low risk outcome group was split using a third classifier (Classifier C) developed in accordance with FIG. 2 with the following parameters and design:
A “label-flip” approach was used, in which training class labels and classifier were simultaneously iteratively refined.
The training class labels for initiation of the iterative refinement were defined so that the patients with lowest TTR times (regardless of event or no event) were in one group and the patients with highest TTR times were in the other group.
The atomic classifiers were k=9 k-nearest neighbor classifiers
Atomic classifiers used 1 or 2 mass spectral features
No feature deselection was used. All 274 features and their pairs were considered in the atomic classifier filtering step.
Filtering was by TTR hazard ratio, with limits 2.5-10.
150,000 dropout iterations were used, each retaining 10 atomic classifiers.
625 test/training split realization were created at each refinement step. For a few realizations, too few atomic classifiers passed filtering for 10 per dropout iteration and master classifiers could not be created. Ensemble averaging was carried out over all generated master classifiers. In particular, the final step of the iterative refinement produced a classifier ensemble averaged over 609 master classifiers.
At each step of the simultaneous iterative refinement process each test/training split realization was randomized to use data from spectra collected on two different mass spectrometer instruments (referred to as “ST1” and “ST100” in this document). This was done to attempt to improve ease of transfer of any resulting test between the two platforms and to help isolate useful information common to multiple data sources.

Results

1. First Split of the Sample Set, Classifier A (Binary Classification)

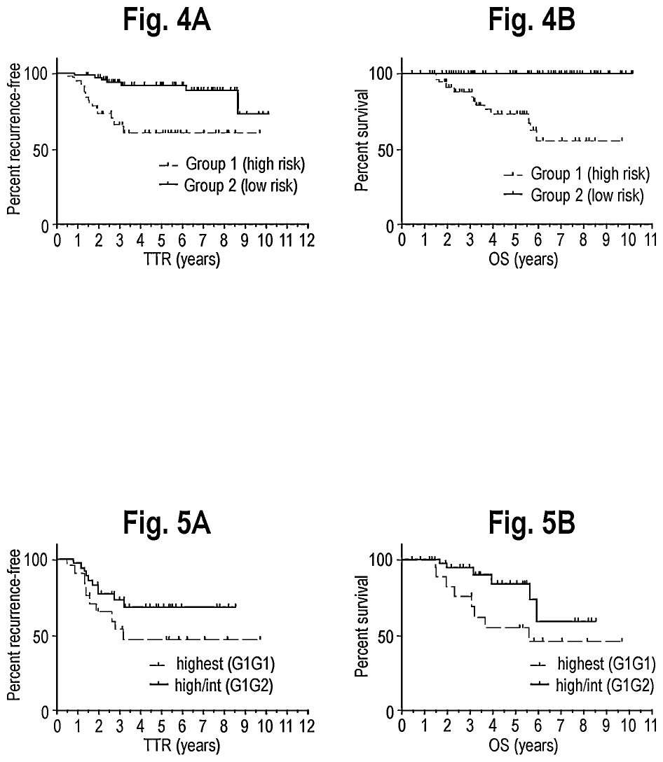

This classifier (“Classifier A”) stratifies the development set into two groups with higher and lower risk of recurrence (or worse and better outcomes). Fifty six patients (45%) were classified to the high risk group and the remaining 68 (55%) to the low risk group. Twenty patients in the high risk group recurred (35% recurrence rate in this group, which includes 74% of the recurrers). Fourteen patients in the high risk group died (25% of this group and 100% of all death events). Time-to-recurrence and overall survival are shown by test classification in FIGS. 4A and 4B. The separation in the plots between the high and low risk groups indicates those patients in the high risk group had significantly worse time to recurrence and overall survival statistics, which is associated with recurrence of the cancer post-surgery.

TABLE 3
Time-to-event comparison by test result

| | HR (95% Cl) | CPH p value | Log-rank p |
|---|---|---|---|
| TTR | 0.21 (0.09-0.50) | p < 0.001 | p < 0.001 |
| OS | *0.07 (0.02-0.20) | — | p < 0.001 |

*Mantel-Haenszel

TABLE 4
Time-to-event landmarks

| | 1 yr | 2 yr | 3 yr | 15 yr |
|---|---|---|---|---|
| Recurrence-free | 95%/99% | 73%/97% | 66%/94% | 60%/92% |
| (high/low) | | | | |
| Survival | 100%/100% | 90%/100% | 87%/1000/ | 73%/100% |
| (high/low) | | | | |

Patient characteristics by test classification are shown in table 5.

TABLE 5
Patient characteristics by binary test classification

| | | High Risk (N = 56) | | Low Risk (N = 68) | | |
|---|---|---|---|---|---|---|
| | | n | (%) | n | (%) | P value |
| Gender | Female | 29 | (52) | 49 | (72) | 0.025 |
| | Male | 27 | (48) | 19 | (28) | |
| Race | White (including Hispanic) | 50 | (89) | 60 | (88) | 0.771 |
| | Other | 5 | (9) | 8 | (12) | (White vs |
| | NA | 1 | (2) | 0 | (0) | Other) |
| Histology | adenocarcinoma | 41 | (73) | 66 | (97) | <0.001 |
| | other | 15 | (27) | 2 | (3) | |
| Smoking | Current | 4 | (7) | 3 | (4) | — |
| Status | Former | 26 | (46) | 28 | (41) | |
| | Never | 3 | (5) | 10 | (15) | |
| | NA* | 23 | (41) | 27 | (40) | |
| TNM T | 1 | 44 | (79) | 58 | (85) | 0.354 |
| | | | | | | (1 vs 2+) |
| | 1a | 18 | | 33 | | |
| | 1b | 11 | | 8 | | |
| | Not specified | 15 | | 17 | | |
| | 2 | 10 | (18) | 9 | (13) | |
| | 2a | 6 | | 6 | | |
| | 2b | 0 | | 0 | | |
| | Not specified | 4 | | 3 | | |
| | 3 | 2 | (4) | 1 | (1) | |
| Pleural | yes | 5 | (9) | 4 | (6) | 0.730 |
| Invasion | no | 51 | (91) | 64 | (94) | |
| LVI | yes | 3 | (5) | 4 | (6) | >0.999 |
| | no | 53 | (95) | 64 | (94) | |
| # nodules | 1 | 53 | (95) | 67 | (99) | 0.327 |
| | | | | | | (1 vs 2) |
| | Size (median, range) | 1.8 | (0.7-8) | 1.6 | (0.5-13) | |
| | 2 | 3 | (5) | 1 | (1) | |
| | Total Size (median, | 4.3 | (3.7-4.5) | 2.9 | | |
| | range) | | | | | |
| Age | Median (range) | 71 | (53-88) | 71 | (46-89) | |

Table 6 shows the ability of the test to predict outcome when adjusted for other patient characteristics.

TABLE 6
Multivariate analysis of TTR adjusting
for other patient characteristics

| TTR: | HR (95% Cl) | | P value |
|---|---|---|---|
| Test (High vs Low) | 0.23 | (0.09-0.57) | 0.002 |
| Age (<70 vs 70+) | 0.73 | (0.32-1.68) | 0.458 |
| T (1 vs 2+) | 2.83 | (1.19-6.78) | 0.019 |
| Gender (M vs F) | 0.74 | (0.33-1.69) | 0.478 |
| Histology | 1.20 | (0.44-3.27) | 0.721 |
| (not adeno vs adeno) | | | |

OS: not done due to lack of events in low risk group

TABLE 7
Types of recurrence by test
classification: high and low

| | high | low |
|---|---|---|
| Distant (metastatic) | 5 | 1 |
| locoregional | 8 | 3 |
| New primary | 7 | 3 |

Reproducibility

Reproducibility was assessed by comparing the test classifications obtained during development by out-of-bag estimate with the results obtained from two reruns of the development sample set on the ST100 and the ST1 machines. The data showed concordance of between 94 and 97 percent on the reruns.

2. Second Split of the Sample Set, Classifier B (Split of High Risk Group from First Stratification)

This classifier (“Classifier B”) stratifies the high risk group defined by the first Classifier (A) into two groups with highest (“highest”) and intermediate (“high/int”) risk of recurrence. Twenty-one patients (37.5% of the high risk group) were classified to the highest risk group and the remaining 35 (62.5%) to the high/int risk group. Ten patients in the highest risk group recurred (48% recurrence rate); ten patients in the high/int group recurred (29% recurrence rate). Eight patients in the highest risk group had an OS event (38% of this group); six patients in the high/int group had an OS event (17%). Time-to-recurrence and overall survival are shown by second split test classification for patients classified as high risk by the first split in FIGS. 5A and 5B.

TABLE 8
Time-to-event comparison of the
highest and intermediate subgroups

| | HR (95% Cl) | | CPH p value | Log-rank p |
|---|---|---|---|---|
| TTR | 0.51 | (0.21-1.22) | 0.129 | 0.122 |
| OS | 0.40 | (0.14-1.15) | 0.090 | 0.079 |

TABLE 9
Time-to-event landmarks

| | 1 yr | 2 yr | 3 yr | 5 yr |
|---|---|---|---|---|
| Recurrence-free | 90%; 97% | 65%; 77% | 53%; 73% | 47%; 69% |
| (highest; high/int) | | | | |
| Survival | 100%; 100% | 82%; 94% | 75%; 94% | 55%; 84% |
| (highest; high/int) | | | | |

TABLE 10
Time-to-event Medians

| | Median TTR (95% CI) | Median OS (95% CI) |
|---|---|---|
| | in years | in years |
| highest | 3.1 (1.5-undefined) | 5.6 (2.3-undefined) |
| high/int | Not reached | Not reached |
| | (3.2-undegined) | (5.6-undefined) |

Patient characteristics by test classification are shown in table 11.

TABLE 11
Patient characteristics of high risk group by second split test classification

| | | Highest Risk | High/Int Risk | |
|---|---|---|---|---|
| | | (N = 21) | (N = 35) | |
| | | n (%) | n (%) | P value |
| Gender | Female | 12 (57) | 17 (49) | 0.589 |
| | Male | 9 (43) | 18 (51) | |
| Race | White (including Hispanic) | 19 (90) | 31 (89) | >0.999 |
| | Other | 2 (10) | 3 (9) | (White vs |
| | NA | 0 (0) | 1 (3) | Other) |
| Histology | adenocarcinoma | 15 (71) | 26 (74) | >0.999 |
| | other | 6 (29) | 9 (26) | |
| Smoking Status | Current | 3 (14) | 1 (3) | — |
| | Former | 10 (48) | 16 (46) | |
| | Never | 3 (14) | 0 (0) | |
| | NA | 5 (24) | 18 (51) | |
| TNM T | 1 | 15 (71) | 29 (83) | 0.335 |
| | 1a | 6 | 12 | (1 vs 2+) |
| | 1b | 3 | 8 | |
| | Not specified | 6 | 9 | |
| | 2 | 6 (29) | 4 (11) | |
| | 2a | 3 | 3 | |
| | 2b | 0 | 0 | |
| | Not specified | 3 | 1 | |
| | 3 | 0 (0) | 2 (6) | |
| Pleural Invasion | yes | 3 (14) | 2 (6) | 0.352 |
| | no | 18 (86) | 33 (94) | |
| LVI | yes | 2 (10) | 1 (3) | 0.549 |
| | no | 19 (90) | 34 (97) | |
| # nodules | 1 | 20 (95) | 33 (94) | >0.999 |
| | Size (median, range) | 2.0 (1.1-5) | 1.7 (0.7-8) | (1 vs 2) |
| | 2 | 1 (5) | 2 (6) | |
| | Total Size | 3.7 | 4.3, 4.5 | |
| Age | Median (range) | 73 (57-88) | 70 (53-87) | |

Table 12 shows ability of the test to predict outcome when adjusted for other patient characteristics.

TABLE 12
Multivariate analysis of TTR and OS for highest vs high/int
classification adjusting for other patient characteristics

| | |
|---|---|
| HR (95% CI) | P value |

TTR:

| | | |
|---|---|---|
| Test (highest vs high/int) | 0.61 (0.25-1.52) | 0.290 |
| Age (<70 vs 70+) | 0.59 (0.23-1.52) | 0.274 |
| T (1 vs 2+) | 2.79 (0.98-7.92) | 0.054 |
| Gender (M vs F.) | 0.97 (0,37-2.52) | 0.943 |
| Histology (not vs adeno) | 1.15 (0.40-3.33) | 0.798 |

OS:

| | | |
|---|---|---|
| Test (highest vs high/int) | 0.46 (0.15-1.43) | 0.179 |
| Age (<70 vs 70+) | 1.28 (0.37-4.38) | 0.700 |
| T (1 vs 2+) | 2.26 (0.62-8.25) | 0.219 |
| Gender (M vs F) | 0.67 (0.19-2.38) | 0.533 |
| Histology (not vs adeno) | 0.66 (0.20-2.26) | 0.513 |

TABLE 13
Type of recurrences by test classification:
highest and high/int

| | highest | high/int |
|---|---|---|
| Distant (metastatic) | 5 | 0 |
| locoregional | 3 | 5 |
| New primary | 2 | 5 |

Reproducibility
Reproducibility was assessed by comparing the test classifications obtained during development by out-of-bag estimate with the results obtained from two reruns of the development sample set on the ST100 and ST1 machines. Concordance was demonstrated at between 91 and 95 percent.

3. Second Split of the Sample Set, Classifier C (Split of Low Risk Group from First Stratification)

This classifier (“Classifier C”) stratifies the low risk group defined by the first classifier (Classifier A) (N=68 with 7 recurrences) into two groups with lowest (“lowest”) and intermediate (“low/int”) risk of recurrence. This classifier was constructed using spectra acquired on the ST1 and ST100 machines. Hence, we can look at out-of-bag estimators for classification of the development set using either ST100 spectra or ST1 spectra.

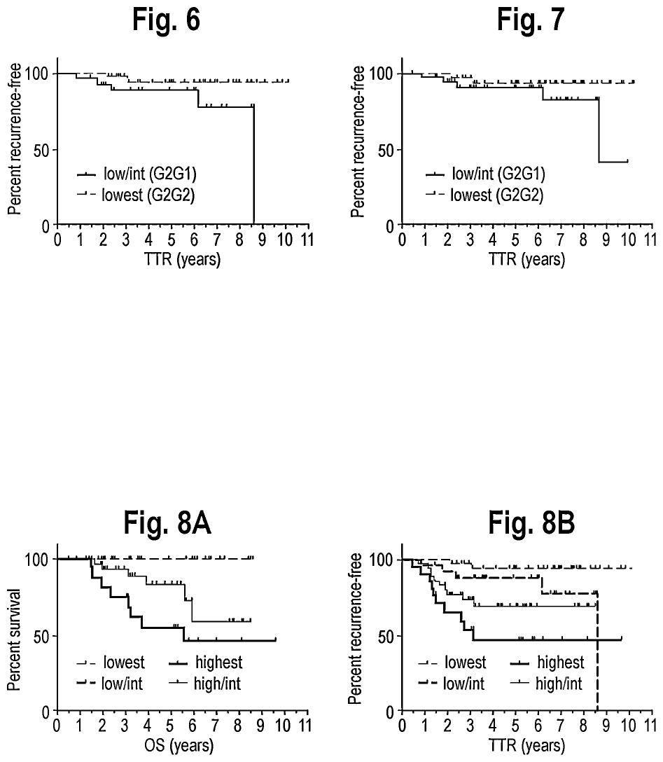

For ST100 out-of-bag analysis, 40 patients (59% of the low risk group) were classified to the lowest risk group and the remaining 28 (41%) to the low/int risk group. Two patients in the lowest risk group recurred (5% recurrence rate); five patients in the low/int group recurred (18% recurrence rate). Time-to-recurrence is shown by second split test classification from ST100 spectra for patients classified as low risk by the first split in FIG. 6.

TABLE 14
TTR comparison of the lowest and low/int subgroups
(ST100 spectra)

| | HR (95% CI) | CPH p value | Log-rank p |
|---|---|---|---|
| TTR | 0.19 (0.04-1.02) | 0.052 | 0.032 |

TABLE 15
Time-to-event landmarks (ST100 spectra)

| | 1 yr | 2 yr | 3 yr | 5 yr |
|---|---|---|---|---|
| Recurrence-free | 96%/100% | 93%/100% | 88%/98% | 88%/94% |
| (low/int; lowest) | | | | |

For ST1 out-of-bag analysis, 33 patients (49% of the low risk group) were classified to the lowest risk group and the remaining 35 (51%) to the low/int risk group. Two patients in the lowest risk group recurred (6% recurrence rate); five patients in the low/int group recurred (14% recurrence rate). Time-to-recurrence is shown by second split test classification from ST1 spectra for patients classified as low risk by the first split in FIG. 7.

TABLE 16
TTR comparison of the lowest and low/int subgroups
(ST1 spectra)

| | HR (95% CI) | CPH p value | Log-rank p |
|---|---|---|---|
| TTR | 0.33 (0.06-1.70) | 0.183 | 0.162 |

TABLE 17
Time-to-event landmarks (STI spectra)

| | 1 yr | 2 yr | 3 yr | 5 yr |
|---|---|---|---|---|
| Recurrence-free | 97%/100% | 94%/100% | 91%/97% | 91%/93% |
| (low/int; lowest) | | | | |

TABLE 18
Patient characteristics of low risk group by second split test classification
(ST100 classifications)

| | | Low/int Risk | Lowest Risk | |
|---|---|---|---|---|
| | | (N = 28) | (N = 40) | |
| | | n (%) | n (%) | P value |
| Gender | Female | 14 (50) | 35 (88) | 0.001 |
| | Male | 14 (50) | 5 (13) | |
| Race | White (including Hispanic) | 21 (75) | 39 (98) | 0.007 |
| | Other | 7 (25) | 1 (3) | (White |
| | | | | vs Other) |
| Histology | adenocarcinorna | 28 (100) | 38 (95) | 0.508 |
| | other | 0 (0) | 2 (5) | |
| Smoking Status | Current | 1 (4) | 2 (5) | — |
| | Former | 12 (43) | 16 (40) | |
| | Never | 4 (14) | 6 (15) | |
| | NA* | 11 (39) | 16 (40) | |
| TNM T | 1 | 23 (82) | 35 (88) | 0.730 |
| | 1a | 16 | 17 | (1 vs 2+) |
| | 1b | 2 | 6 | |
| | Not specified | 5 | 12 | |
| | 2 | 4 (14) | 5 (13) | |
| | 2a | 2 | 4 | |
| | 2b | 0 | 0 | |
| | Not specified | 2 | 1 | |
| | 3 | 1 (4) | 0 (0) | |
| Pleural Invasion | yes | 2 (7) | 2 (5) | >0.999 |
| | no | 26 (93) | 38 (95) | |
| LVI | yes | 2 (7) | 2 (5) | >0.999 |
| | no | 26 (93) | 38 (95) | |
| # nodules | 1 | 28 (100) | 39 (98) | >0.999 |
| | Size (median, range) | 1.8 (0.7-4) | 1.5 (0.5-13) | (1 vs 2) |
| | 2 | 0 (0) | 1 (3) | |
| | Total Size | — | 2.9 | |
| Age | Median (range) | 71 (53-88) | 70 (46-89) | |

TABLE 19
Types of recurrences by test classification:
lowest and low/int

| | Low/int | lowest |
|---|---|---|
| Distant (metastatic) | 1 | 0 |
| locoregional | 2 | 1 |
| New primary | 2 | 1 |

Reproducibility

Reproducibility was assessed by comparing the test classifications obtained during development for the ST100 spectra by out-of-bag estimate with the results obtained from two reruns of the development sample set on the ST100 and for the rerun of the development sample set on the ST1. To compare between the results for the ST1 original run (also used in development) and the ST100 original run, out-of-bag estimates were used for both classifications. The data showed concordance of between 87 and 91 percent.

Four-Way Split of the Cohort

A procedure for combining the three classifiers in a hierarchical manner to give a four-way classification of patients is illustrated in FIG. 3. The procedure of FIG. 3 is implemented in software in a laboratory computer that executes the classification procedure of Classifiers A, B and C. Spectra are first classified by the “first split” classifier (Classifier A) to generate a high risk or low risk classification. Patients with spectra classified as high risk are then classified using the second split classifier (classifier B) for the high risk group to yield a classification of highest or high/int. Patients with spectra classified as low risk are then classified using the second split classifier (Classifier C) for the low risk group to yield a classification of lowest or low/int. This is shown schematically in FIG. 3.

TABLE 20
Patient characteristics by lowest, low/int, high/int and highest test classifications

| | | Lowest | Low/int | High/int | Highest |
|---|---|---|---|---|---|
| | | Risk | Risk | Risk | Risk |
| | | (N = 40) | (N = 28) | (N = 35) | (N = 21) |
| | | n (%) | n (%) | n (%) | n (%) |
| Gender | Female | 35 (88) | 14 (50) | 17 (49) | 12 (57) |
| | Male | 5 (13) | 14 (50) | 18 (51) | 9 (43) |
| Race | White (including Hispanic) | 39 (98) | 21 (75) | 31 (89) | 19 (90) |
| | Other | 1 (3) | 7 (25) | 3 (9) | 2 (10) |
| | NA | 0 (0) | 0 (0) | 1 (3) | 0 (0) |
| Histology | adenocarcinoma | 38 (95) | 28 (100) | 26 (74) | 15 (71) |
| | other | 2 (5) | 0 (0) | 9 (26) | 6 (29) |
| Smoking Status | Current | 2 (5) | 1 (4) | 1 (3) | 3 (14) |
| | Former | 16 (40) | 12 (43) | 16 (46) | 10 (48) |
| | Never | 6 (15) | 4 (14) | 0 (0) | 3 (14) |
| | NA* | 16 (40) | 11 (39) | 18 (51) | 5 (24) |
| TNM T | 1 | 35 (88) | 23 (82) | 29 (83) | 15 (71) |
| | 1a | 17 | 16 | 12 | 6 |
| | 1b | 6 | 2 | 8 | 3 |
| | Not specified | 12 | 5 | 9 | 6 |
| | 2 | 5 (13) | 4(14) | 4 (11) | 6 (29) |
| | 2a | 4 | 2 | 3 | 3 |
| | 2b | 0 | 0 | 0 | 0 |
| | Not specified | 1 | 2 | 1 | 3 |
| | 3 | 0 (0) | 1 (4) | 2 (6) | 0 (0) |
| Pleural | yes | 2 (5) | 2 (7) | 2 (6) | 3 (14) |
| Invasion | no | 38 (95) | 26 (93) | 33 (94) | 18 (86) |
| LVI | yes | 2 (5) | 2 (7) | 1 (3) | 2 (10) |
| | no | 38 (95) | 26 (93) | 34 (97) | 19 (90) |
| # nodules | 1 | 39 (98) | 28 (100) | 33 (94) | 20 (95) |
| | Size (median, range) | 1.5 (0.5-13) | 1.8 (0.7-4) | 1.7 (0.7-8) | 2.0 (1.1-5) |
| | 2 | 1 (3) | 0 (0) | 2 (6) | 1 (5) |
| | Total Size | 2.9 | — | 4.3, 4.5 | 3.7 |
| Age | Median (range) | 70 (46-89) | 71 (53-88) | 70 (53-87) | 73 (57-88) |

Time-to-recurrence and overall survival for the whole development cohort stratified by four-way test classification are shown in FIGS. 8A and 8B. In FIG. 8A, the low/int and lowest plots are superimposed as there were no events in either group.

TABLE 21
Time-to-event landmarks summary

| Recurrence-free | 1 yr | 2 yr | 3 yr | 5 yr |
|---|---|---|---|---|
| highest | 90% | 65% | 53% | 47% |
| High/int | 97% | 77% | 73% | 69% |
| Low/int | 96% | 93% | 88% | 88% |
| lowest | 100% | 100% | 98% | 94% |
| Survival | 1 yr | 2 yr | 3 yr | 5 yr |
| highest | 100% | 82% | 75% | 55% |
| High/int | 100% | 94% | 94% | 84% |
| Low/int | 100% | 100% | 100% | 100% |
| lowest | 100% | 100% | 100% | 100% |

TABLE 22
Types of recurrences by test classifications:
lowest, low/int, high/int, and highest

| | highest | high/int | low/int | lowest |
|---|---|---|---|---|
| Distant (metastatic) | 5 | 0 | 1 | 0 |
| locoregional | 3 | 5 | 2 | 1 |
| New primary | 2 | 5 | 2 | 1 |

Reproducibility

Reproducibility of the 4 way classification of FIG. 3 was assessed relative to the ST100 classification obtained with out-of-bag estimates for all three classifiers. ST1 classifications were generated using majority vote for Classifiers A and B and out-of-bag estimates for Classifier C. Majority vote classifications were used for all three classifiers. Concordance of between 85% and 90% was obtained.

In terms of a practical test, in one embodiment the classification in the hierarchical manner as shown in FIG. 3 is performed. The split of the low risk group in this setting (stage 1A/B patients), aside from the prediction of low risk of recurrence, could have value in a clinical setting, for example by possibly excluding patients from aggressive treatment. With respect to the split of the high risk group by Classifier B, it is useful to have a kind of level of risk, and it could differentiate by the type of treatment. While in theory one could include clinical factors to affect classification results (for example by including them in the feature space during classifier generation), one could also use the intermediate classification results to affect choice of treatment. For example, knowing prognosis before surgery could affect surgical planning, and possibly include neo-adjuvant therapies. Additionally, one could also use post-surgery samples to possibly refine the tests, for example by repeating the classification per the schema of FIG. 3 and using new test results to further guide treatment.

As another alternative, it is possible that a test could be performed using only Classifiers A, or the combination of Classifiers A and B in the schema of FIG. 3. This embodiment would be performed for example seeking to only identify if the patient was at the highest risk of recurrence (and only such patients are guided to more aggressive treatments). If the patient tests “low risk” by classifier A, no further stratification using classifier C is performed. If the patient is classified as “high risk” by classifier A then the sample is subject to classification by classifier B, and if that classifier produces the “highest risk” classification label for the sample the patient is guided towards more aggressive treatment for the cancer.

Section 5: Association of Test Classifications with Biological Processes Using Protein Set Enrichment Analysis (PSEA)

When building tests using the procedure of FIG. 3, it is not essential to be able to identify which proteins correspond to which mass spectral features in the MALDI TOF spectrum or to understand the function of proteins correlated with these features. Whether the process produces a useful classifier depends entirely on classifier performance on the development set and how well the classifier performs when classifying new sample sets. However, once a classifier has been developed it may be of interest to investigate the proteins or function of proteins which directly contribute to, or are correlated with, the mass spectral features used in the classifier. In addition, it may be informative to explore protein expression or function of proteins, measured by other platforms, that are correlated with the test classification groups.

We used a method known as Gene Set Enrichment Analysis (GSEA) applied to protein expression data, which is referred to as Protein Set Enrichment Analsyis (PSEA). Background information on this method is set forth in Mootha, et al., PGC-1α-responsive genes involved in oxidative phosphorylation are coordinately downregulated in human diabetes. Nat Genet. 2003; 34(3):267-73 and Subramanian, et al., Gene set enrichment analysis: A knowledge-based approach for interpreting genome-wide expression profiles. Proc Natl Acad Sci USA 2005; 102(43): 15545-50, the content of which are incorporated by reference herein. Further details are explained at length in the patent literature, see U.S. Pat. No. 10,007,766, therefore a detailed discussion is omitted for the sake of brevity. High risk vs low risk (Classifier A)

Classifier A was applied to two sample sets with matched mass spectral and protein panel data (see the discussions in the literature cited above) and the resulting test classifications used as the phenotype for set enrichment analysis. These results were then merged to produce an overall p value of association with a set of 26 biological processes. These results are tabulated below, together with the false discovery rates (FDRs) calculated by the Benjamini-Hochberg method.

TABLE 23
PSEA p values and FDR for high risk vs low risk phenotypes

| Biological Process | p value | FDR |
|---|---|---|
| Acute phase response | 0.00002 | <0.001 |
| Complement activation (narrow definition) | 0.00004 | <0.001 |
| Acute inflammatory response | 0.00032 | <0.005 |
| Wound healing (broad definition) | 0.00045 | <0.005 |
| Complement activation (broad definition) | 0.00109 | <0.01 |
| Wound healing (narrow definition) | 0.00156 | <0.01 |
| Innate immune response | 0.10137 | <0.40 |
| Cellular component of morphogenesis | 0.10572 | <0.40 |
| Chronic Inflammatory response | 0.15376 | <0.50 |
| Immune tolerance | 0.24747 | <0.60 |
| Type2 immune response | 0.26399 | <0.60 |
| Immune tolerance and suppression | 0.28140 | <0.60 |
| Type17 immune response | 0.28623 | <0.60 |
| Glycolysis | 0.31633 | <0.60 |
| Interferon type 1 | 0.32695 | <0.60 |
| Type1 immune response | 0.35829 | <0.60 |
| B cell mediated immunity | 0.42120 | <0.65 |
| Extracellular matrix organization | 0.47315 | <0.65 |
| Angiogenesis | 0.48261 | <0.65 |
| Cytokine production involved in | 0.50037 | <0.65 |
| immune response | | |
| NK cell mediated immunity | 0.52331 | <0.65 |
| Hypoxia | 0.53669 | <0.65 |
| T cell mediated immunity | 0.55349 | <0.65 |
| Behavior | 0.58693 | <0.65 |
| Interferon Gamma | 0.62726 | <0.70 |
| Epithelial mesenchymal transition | 0.68697 | <0.70 |

Highest vs High/Int (Classifier B)

Classifiers A and B were applied to the two sample sets with matched mass spectral and protein panel data. Samples classified as highest risk and high/int risk were identified and these classifications used as the phenotype for set enrichment analysis. PSEA was carried out and results were then merged to produce an overall p value of association with a set of 26 biological processes. These results are tabulated below, together with the false discovery rates (FDRs) calculated by the Benjamini-Hochberg method.

TABLE 24
PSEA p values and FDR for highest risk vs high/int risk phenotypes

| Biological Process | p value | FDR |
|---|---|---|
| Complement activation (narrow definition) | 0.00057 | <0.05 |
| Acute phase response | 0.00491 | <0.10 |
| Complement activation (broad definition) | 0.00654 | <0.10 |
| Acute inflammatory response | 0.02804 | <0.20 |
| Chronic Inflammatory response | 0.09403 | <0.50 |
| Innate immune response | 0.15714 | <0.70 |
| Epithelial mesenchymal transition | 0.19526 | <0.70 |
| Immune tolerance | 0.22519 | <0.70 |
| NK cell mediated immunity | 0.25293 | <0.70 |
| Hypoxia | 0.27842 | <0.70 |
| Immune tolerance and suppression | 0.28199 | <0.70 |
| Wound healing (narrow definition) | 0.49899 | <1.00 |
| Type1 immune response | 0.51219 | <1.00 |
| Angiogenesis | 0.54609 | <1.00 |
| Wound healing (broad definition) | 0.57156 | <1.00 |
| Behavior | 0.57818 | <1.00 |
| Cytokine production involved in immune response | 0.59935 | <1.00 |
| Type17 immune response | 0.73064 | <1.00 |
| Interferon Gamma | 0.74699 | <1.00 |
| Extracellular matrix organization | 0.80689 | <1.00 |
| B cell mediated immunity | 0.81926 | <1.00 |
| Interferon type 1 | 0.82980 | <1.00 |
| Type2 immune response | 0.83416 | <1.00 |
| T cell mediated immunity | 0.88350 | <1.00 |
| Cellular component of morphogenesis | 0.88643 | <1.00 |
| Glycolysis | 0.91085 | <1.00 |

Highest vs Lowest Risk

Classifiers A, B, and C were applied to the sample sets. Samples classified as highest risk and lowest risk were identified and these classifications used as the phenotype for set enrichment analysis. PSEA was carried out and the results were then merged to produce an overall p value of association with a set of 26 biological processes. These results are tabulated below, together with the false discovery rates (FDRs) calculated by the Benjamini-Hochberg method.

TABLE 25
PSEA p values and FDR for highest risk vs
lowest risk phenotypes

| Biological Process | p value | FDR |
|---|---|---|
| Extracellular matrix organization | 0.01728 | <0.30 |
| Acute phase response | 0.01921 | <0.30 |
| Innate immune response | 0.02413 | <0.30 |
| Wound healing (narrow definition) | 0.06033 | <0.40 |
| Complement activation (narrow definition) | 0.06794 | <0.40 |
| Acute inflammatory response | 0.07731 | <0.40 |
| Interferon type 1 | 0.09660 | <0.40 |
| Epithelial mesenchymal transition | 0.20931 | <0.60 |
| Type1 immune response | 0.30997 | <0.60 |
| Type2 immune response | 0.32510 | <0.60 |
| Interferon Gamma | 0.33278 | <0.60 |
| Wound healing (broad definition) | 0.34334 | <0.60 |
| Hypoxia | 0.34951 | <0.60 |
| Behavior | 0.34963 | <0.60 |
| B cell mediated immunity | 0.35442 | <0.60 |
| Cytokine production involved in | 0.35724 | <0.60 |
| immune response | | |
| Cellular component of morphogenesis | 0.44647 | <0.70 |
| T cell mediated immunity | 0.46747 | <0.70 |
| Type17 immune response | 0.55694 | <0.80 |
| Complement activation (broad definition) | 0.56869 | <0.80 |
| Chronic Inflammatory response | 0.65442 | <0.90 |
| Immune tolerance | 0.71257 | <0.90 |
| Glycolysis | 0.80969 | <0.90 |
| Angiogenesis | 0.81055 | <0.90 |
| NK cell mediated immunity | 0.86104 | <0.90 |
| Immune tolerance and suppression | 0.94283 | <1.00 |

Low/int vs Lowest Risk

Classifiers A and C were applied to the sample sets. Samples classified as lowest risk and low/int risk were identified and these classifications used as the phenotype for set enrichment analysis. PSEA was carried out and the results were then merged to produce an overall p value of association with a set of 26 biological processes. These results are tabulated below, together with the false discovery rates (FDRs) calculated by the Benjamini-Hochberg method.

TABLE 26
PSEA p values and FDR for low/int risk vs
lowest risk phenotypes

| Biological Process | p value | FDR |
|---|---|---|
| Complement activation (narrow definition) | 0.00218 | <0.05 |
| Complement activation (broad definition) | 0.00296 | <0.05 |
| Acute inflammatory response | 0.00380 | <0.05 |
| Immune tolerance | 0.01048 | <0.10 |
| Acute phase response | 0.02158 | <0.10 |
| Wound healing (broad definition) | 0.02202 | <0.10 |
| Glycolysis | 0.02473 | <0.10 |
| Innate immune response | 0.03251 | <0.20 |
| Extracellular matrix organization | 0.03518 | <0.20 |
| Interferon type 1 | 0.04380 | <0.20 |
| Cytokine production involved in | 0.12235 | <0.30 |
| immune response | | |
| Immune tolerance and suppression | 0.12601 | <0.30 |
| Angiogenesis | 0.21818 | <0.50 |
| Hypoxia | 0.24927 | <0.50 |
| Wound healing (narrow definition) | 0.32864 | <0.60 |
| Type1 immune response | 0.37013 | <0.60 |
| Cellular component of morphogenesis | 0.39397 | <0.60 |
| Type2 immune response | 0.42161 | <0.60 |
| T cell mediated immunity | 0.44928 | <0.60 |
| Chronic Inflammatory response | 0.46653 | <0.60 |
| Type17 immune response | 0.48864 | <0.60 |
| B cell mediated immunity | 0.49909 | <0.60 |
| Epithelial mesenchymal transition | 0.87725 | <1.00 |
| Interferon Gamma | 0.87869 | <1.00 |
| Behavior | 0.90281 | <1.00 |
| NK cell mediated immunity | 0.97907 | <1.00 |

Section 6: Laboratory Testing Environment

We further contemplate a laboratory test center for conducting tests on blood-based samples to assess the risk of an early stage NSCLC patient of recurrence of the cancer. The lab test center is configured as per Example 5 and FIG. 15 of the prior U.S. Pat. No. 10,007,766, and that description is incorporated by reference herein, The laboratory test center or system includes a mass spectrometer (e.g., MALDI time of flight) and a general purpose computer system having a CPU implementing Classifier A or alternatively a hierarchical arrangement of classifiers (see FIG. 3) coded as machine-readable instructions implementing final classifiers (A, optionally B and C) developed using the procedure of FIG. 2, including classification weights, miniClassifiers definitions passing filtering, etc., program code implementing a hierarchical classification procedure as per FIG. 3 or 14, and a memory storing a reference mass spectral data set including a feature table of class-labeled mass spectrometry data from NSCLC patients used to develop the classifiers per FIG. 2, including feature values of the features listed in Appendix A. This reference mass spectral data set forming the feature table will be understood to be the mass spectral data (integrated intensity values of predefined features, Appendix A) of a set of spectra which were used to generate the classifiers during classifier development.

Conclusions

We were able to create a suite of three classifiers stratifying patients with early stage lung cancer by risk of recurrence. Seventeen percent of patients in the development set were assigned to the highest risk group, 23% to the high/intermediate risk group, 28% to the low/intermediate risk group and 32% to the lowest risk group. The percentage of patients recurrence-free at two years varied from 65% in the highest risk group to 100% in the lowest risk group; the percentage of patients alive at five years was 55% in the highest risk group and 100% in the lowest risk group. Although sample sizes were too small, given the few events, for statistical significance except in the first split of the cohort into low and high risk groups, multivariate analysis indicated that hazard ratios for all three classifiers were stable on adjustment for other patient characteristics. It is noteworthy that the tests were able to stratify all three kinds of recurrence: distant, locoregional and new primary.

Protein set enrichment analysis indicated that test classifications were associated with acute phase response, complement activation, acute inflammatory response and wound healing. Immune tolerance and glycolytic processes could also be potentially relevant. These observations, together with our experience showing the relevance of complement, wound healing, acute phase response and acute inflammatory response in metastatic cancer treated with immunotherapies and the fact that the classifiers are able to stratify risk of new primary lesions, could indicate that the test is accessing information on the host's immune response to cancer,

Reproducibility of the test classifications was very good and the test transferred well between mass spectrometer instruments. The preliminary assessment of reproducibility of the four-way classifications was 85% or better.

Section 7: Redevelopment of Test Using Additional Samples from Validation Set

We decided to redevelop the test described above. As a sample development set we combined the original development set of samples described in Section 1 above with some initial validation samples we had from the same source. As there are relatively few recurrers in this indication, we needed to boost the dataset to improve the reliability of the test beyond a first split of the dataset, namely the second and third splits of the sample sets by classifiers B and C. This section will describe this redevelopment work, including a new ternary or three-way hierarchical combination of the classifiers A, B and C, see FIG. 14.

Sample Set Description

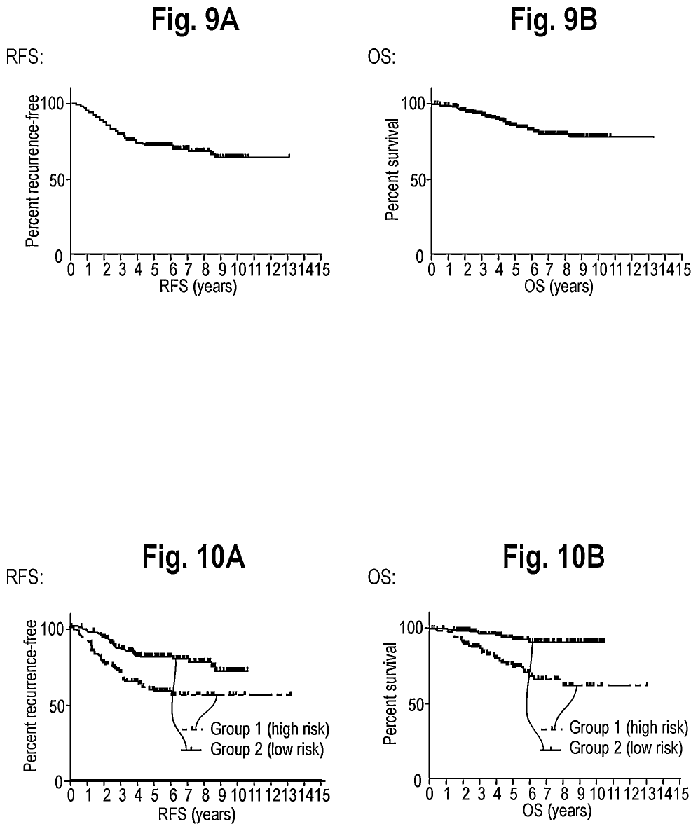

Serum samples taken pre-surgery were available from 314 patients with Stage IA or IB NSCLC. No patients received adjuvant therapy following surgery. Median follow up of these patients was 4.92 years. Patient characteristics are summarized in Table 27. FIGS. 9A and 9B show the recurrence-free survival (RFS) and overall survival (OS) for the cohort, respectively. Recurrence was identified in 80 patients (25%). Of these recurrences, 27 (34%) were new primaries, 32 (40%) were locoregional recurrences, and 21 (26%) were distant recurrences. A further 5 patients died without documented recurrence and these deaths were considered events for the RFS endpoint. Death was observed for 44 patients (14%); however, date of death was unknown for 3 of these patients (IDs 745, 1147, 1513), who were therefore censored for survival at last follow up date.

TABLE 27
Patient characteristics for the development cohort

| | | n (%) |
|---|---|---|
| Gender | Female | 197 (63) |
| | Male | 117 (37) |
| Race | White (including | 286 (91) |
| | Hispanic) | |
| | Other | 27 (9) |
| | NA | 1 (<1) |
| Histology | Adenocarcinoma | 267 (85) |
| | Other | 46 (15) |
| | NA | 1 (<1) |
| TNM T | 1 | 245 (78) |
| | 1a | 113 |
| | 1b | 56 |
| | Not specified | 76 |
| | 2 | 63 (20) |
| | 2a | 45 |
| | 2b | 0 |
| | Not specified | 18 |
| | 3 | 6 (2) |
| Pleural Invasion | Yes | 33 (11) |
| | no | 280 (89) |
| | NA | 1 (<1) |
| LVI | yes | 31 (10) |
| | No | 283 (90) |
| Size nodules | Median (range) | 1.7 |
| | | (0.5-13) |
| Age | Median (range) | 69 |
| | | (46-92) |

Fifteen recurrences were observed within 1 year (4 new primary, 5 locoregional, 6 systemic), a further 24 were observed between 1 and 2 years after surgery (5 distant, 13 locoregional, and 6 new primaries).

TABLE 28
Time-to-event landmarks for the whole cohort

| | 1 yr | 2 yr | 3 yr | 4 yr | 5 yr | 10 yr |
|---|---|---|---|---|---|---|
| Recurrence-free | 95% | 86% | 80% | 74% | 71% | 64% |
| Survival | 99% | 95% | 93% | 89% | 86% | 79% |

Sample preparation and spectral acquisition was the same as described previously.

Spectral processing was the same as described previously.

Classifier development for classifiers A, B and C used the “Diagnostic Cortex” procedure of FIG. 2, described in detail previously.

First split of the sample set (Classifier A) into High and Low risk groups.

A first split of the 314 sample set was achieved using a Diagnostic Cortex classifier (Classifier A) with the following parameters and design:
A “label-flip” approach was used, in which training class labels and classifier were simultaneously iteratively refined.
The training class labels for initiation of the iterative refinement were defined so that the patients with lowest RFS times (regardless of event or no event) were in one group and the patients with highest RFS times were in the other group.
The atomic classifiers were k=9 k-nearest neighbor classifiers
Atomic classifiers used 1 or 2 mass spectral features simultaneously.
No feature deselection was used, All 274 features and their pairs were considered in the atomic classifier filtering step.
Filtering was by RFS hazard ratio, with limits 2.5-10.
100,000 dropout iterations were used, each retaining 10 atomic classifiers.
375 test/training splits were ensemble averaged.
   The performance of this Classifier A will described below in conjunction with FIG. 10A and 10B in the Results section.

Classifier B: a split of the poor outcome group (“high risk”) resulting from the first split produced by Classifier A

The first split of the sample set produced by Classifier A resulted in a poor outcome group (i.e., those patients with a high risk of recurrence) of 137 patients, with 47 recurrers (34%).

To further stratify by outcome, this poor outcome group was further split using a Diagnostic Cortex classifier (classifier B) with the following parameters and design:
A “label-flip” approach was used, in which training class labels and classifier were simultaneously iteratively refined.
The training class labels for initiation of the iterative refinement were defined so that the patients with lowest RFS times (regardless of event or no event) were in one group and the patients with highest RFS times were in the other group.
The atomic classifiers were k=9 k-nearest neighbor classifiers
Atomic classifiers used 1 or 2 mass spectral features simultaneously.
No feature deselection was used. All 274 features and their pairs were considered in the atomic classifier filtering step,
Filtering was by RFS hazard ratio, with limits 2.2-10.
100,000 dropout iterations were used, each retaining 10 atomic classifiers.
375 test/training splits were ensemble averaged.

The performance of this Classifier B is described below in the Results section.

Classifier C a split of the good outcome group from the first split produced by Classifier A.

The first split of the sample set produced by Classifier A resulted in a good outcome group (i.e., a group of patients with a low risk of recurrence) of 177 patients, with 33 recurrers (19%).

To further stratify by outcome, this good outcome group was split using a Diagnostic Cortex classifier (Classifier C) with the following parameters and design:
A “label-flip” approach was used, in which training class labels and classifier were simultaneously iteratively refined.
The training class labels for initiation of the iterative refinement were defined so that the patients with lowest RFS times (regardless of event or no event) were in one group and the patients with highest RFS times were in the other group.
The atomic classifiers were k=9 k-nearest neighbor classifiers.
Atomic classifiers used 1 or 2 mass spectral features simultaneously.
No feature deselection was used. All 274 features and their pairs were considered in the atomic classifier filtering step.
Filtering was by RFS hazard ratio, with limits 2.2-10.
100,000 dropout iterations were used, each retaining 10 atomic classifiers.
375 test/training split realization were created at each refinement step.

Redevelopment Results 1. First Split of the Sample Set (Binary Classification), Classifier A

This classifier (“Classifier A”) stratifies the development set into two groups with higher and lower risk of recurrence (or, equivalently, worse/poor and better/good outcomes). 137 patients (44%) were classified to the high risk group and the remaining 177 (56%) to the low risk group. Forty-seven patients in the high risk group recurred (34% recurrence rate in this group, which includes 59% of the recurrers). Thirty-one patients in the high risk group died (23% of this group and 76% of all death events). Recurrence-free survival and overall survival are shown by test classification in FIGS. 10A and 10B.

TABLE 29
Time-to-event comparison by binary test classification

| | HR (95% CI) | CPH p value | Log-rank p |
|---|---|---|---|
| RFS | 0.42 (0.27-0.65) | p < 0.001 | p < 0.001 |
| OS | 0.21 (0.10-0.43) | p < 0.001 | p < 0.001 |

TABLE 30
Time-to-event landmarks

| | 1 yr | 2 yr | 3 yr | 5 yr |
|---|---|---|---|---|
| Recurrence-free | 92%/98% | 78%/93% | 71%/86% | 59%/81% |
| (high/low) | | | | |
| Survival | 99%/100% | 91%/99% | 87%/97% | 76%/93% |
| (high/low) | | | | |

Patient characteristics by test classification are shown in table 31.

TABLE 31
Patient characteristics by binary test classification

| | | High Risk | Low Risk | |
|---|---|---|---|---|
| | | (N = 137) | (N = 177) | |
| | | n (%) | n (%) | P value |
| Gender | Female | 67 (49) | 130 (73) | <0.001 |
| | Male | 70 (51) | 47 (27) | |
| Race | White (including | 125 (91) | 161 (91) | 0.841 |
| | Hispanic) | | | |
| | Other | 11 (8) | 16 (9) | (White vs |
| | NA | 1 (1) | 0 (0) | Other) |
| Histology | adenocarcinoma | 108 (79) | 159 (90) | 0.015 |
| | other | 28 (20) | 18 (10) | (Adeno |
| | NA | 1 (1) | 0 (0) | vs Other) |
| TNM T | 1 | 102 (74) | 143 (81) | 0.216 |
| | 1a | 42 | 71 | (1 vs 2+) |
| | 1b | 29 | 27 | |
| | Not specified | 31 | 45 | |
| | 2 | 32 (23) | 31 (18) | |
| | 2a | 23 | 22 | |
| | 2b | 0 | 0 | |
| | Not specified | 9 | 9 | |
| | 3 | 3 (2) | 3 (2) | |
| Pleural | yes | 18 (13) | 15 (8) | 0.199 |
| Invasion | no | 119 (87) | 161 (91) | (yes vs |
| | NA | 0 (0) | 1 (1) | no) |
| LVI | yes | 11 (8) | 20 (11) | 0.446 |
| | no | 126 (92) | 157 (89) | |
| Age | Median (range) | 72 | 68 | <0.001 |
| | | (46-92) | (46-89) | |
| Nodule | Median(range) | 1.8 | 1.6 | 0.034 |
| Size | | (0.6-5) | (0.5-13) | |

Tables 32 and 33 show the ability of the test to predict RFS and OS when adjusted for other patient characteristics.

TABLE 32
Multivariate analysis of RFS adjusting
for other patient characteristics
RFS

| | HR (95% CI) | P value |
|---|---|---|
| Test (High vs Low) | 0.49 (0.31-0.78) | 0.003 |
| Age (<70 vs 70+) | 0.88 (0.56-1.38) | 0.582 |
| T (1 vs 2+) | 2.62 (1.66-4.12) | <0.001 |
| Gender (M vs F) | 0.59 (0.37-0.94) | 0.026 |
| Histology (not adeno vs | 1.31 (0.77-2.22) | 0.321 |
| adeno) | | |

TABLE 33
Multivariate analysis of OS adjusting for
other patient characteristics
OS

| | HR (95% CI) | P value |
|---|---|---|
| Test (High vs Low) | 0.27 (0.13-0.57) | 0.001 |
| Age (<70 vs 70+) | 1.28 (0.66-2.50) | 0.468 |
| T (1 vs 2+) | 3.50 (1.86-6.59) | <0.001 |
| Gender (M vs F) | 0.62 (0.31-1.23) | 0.172 |
| Histology (not adeno vs | 1.60 (0.78-3.31) | 0.203 |
| adeno) | | |

TABLE 34
Types of recurrence by test classification: High and Low

| | High (N = 137) | Low (N = 177) |
|---|---|---|
| Distant (metastatic) | 14 | 7 |
| Locoregional | 19 | 13 |
| New primary | 14 | 13 |

Reproducibility was assessed by comparing the test classifications obtained during development by out-of-bag estimate with the results obtained from two reruns of 124 samples from the development sample set on the ST100. The results showed a concordance of test classifications of 94% and 89%

2. Second Split of the Sample Set (Split Of High Risk Group from First Stratification), Classifier B

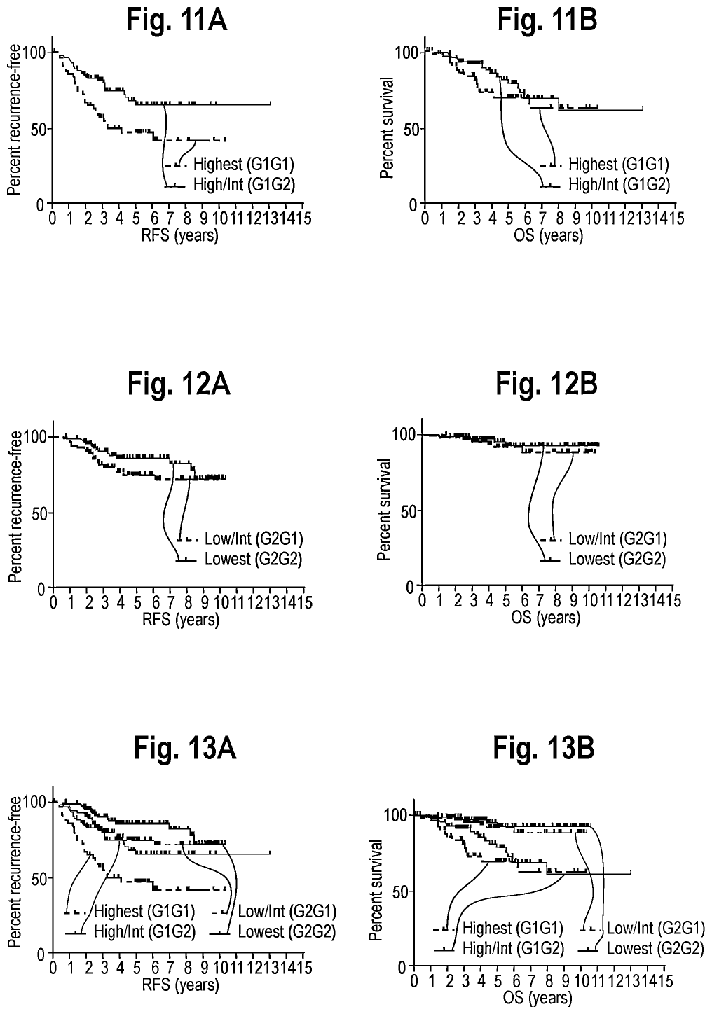

This classifier (“Classifier B”) stratifies the high risk group defined by the first classifier (N=137) into two groups with highest (“highest”) and intermediate (“high/int”) risk of recurrence. Fifty-six patients (41% of the high risk group) were classified to the highest risk group and the remaining 81 (59%) to the high/int risk group. Twenty-six patients in the highest risk group had a documented recurrence (46% recurrence rate); twenty-one patients in the high/int group had a documented recurrence (26% recurrence rate). Fourteen patients in the highest risk group had an OS event (25% of this group); seventeen patients in the high/int group had an OS event (21%). Recurrence-free and overall survival are shown by second split test classification for patients classified as high risk by the first split in FIG. 11A and 11B, respectively.

TABLE 35
Time-to-event comparison of the highest
and high/int subgroups

| | HR (95% CI) | CPH p value | Log-rank p |
|---|---|---|---|
| RFS | 0.47 (0.27-0.82) | 0.008 | 0.006 |
| OS | 0.69 (0.34-1.40) | 0.300 | 0.297 |

TABLE 36
Time-to-event landmarks

| | 1 yr | 2 yr | 3 yr | 5 yr |
|---|---|---|---|---|
| Recurrence-free | 85%/96% | 67%/85% | 57%/81% | 47%/68% |
| (highest; high/int) | | | | |
| Survival | 98%/99% | 86%/94% | 81%/91% | 69%/81% |
| (highest; high/int) | | | | |

TABLE 37
Time-to-event Medians

| | Median RFS (95% CI) | Median OS (95% CI) |
|---|---|---|
| | in years | in years |
| highest | 3.3 (2.5-undefined) | Not reached (6.3-undefined) |
| high/int | Not reached (undefined) | Not reached (8.0-undefined) |

Patient characteristics by test classification are shown in table 38.

TABLE 38
Patient characteristics of high risk group by
second split test classification

| | | Highest | High/Int | |
|---|---|---|---|---|
| | | Risk | Risk | |
| | | (N = 56) | (N = 81) | |
| | | n (%) | n (%) | P value |
| Gender | Female | 34 (61) | 33 (41) | 0.025 |
| | Male | 22 (39) | 48 (59) | |
| Race | White (including | 50 (89) | 75 (93) | 0.360 |
| | Hispanic) | | | |
| | Other | 6 (11) | 5 (6) | (White |
| | NA | 0 (0) | 1 (1) | vs |
| | | | | Other) |
| Histology | Adenocarcinoma | 44 (79) | 64 (79) | 0.833 |
| | Other | 12 (21) | 16 (20) | (Adeno |
| | NA | 0 (0) | 1 (1) | vs |
| | | | | Other |
| TNM T | 1 | 37 (66) | 65 (80) | 0.074 |
| | 1a | 16 | 26 | (1 vs |
| | 1b | 9 | 20 | 2+) |
| | Not specified | 12 | 19 | |
| | 2 | 18 (32) | 14 (17) | |
| | 2a | 13 | 10 | |
| | 2b | 0 | 0 | |
| | Not specified | 5 | 4 | |
| | 3 | 1 (2) | 2 (2) | |
| Pleural | yes | 10 (18) | 8 (10) | 0.204 |
| Invasion | No | 46 (82) | 73 (90) | |
| LVI | yes | 7 (13) | 4 (5) | 0.123 |
| | no | 49 (88) | 77 (95) | |
| Size | Median (range) | 1.9 | 1.8 | 0.393 |
| nodules | | (0.60-5) | (0.7-3.6) | |
| Age | Median (range) | 71 | 73 | 0.529 |
| | | (46-92) | (49-88) | |

Tables 39 and 40 show the ability of the test (highest vs high/int) to predict outcome when adjusted for other patient characteristics.

TABLE 39
Multivariate analysis of RFS adjusting for
other patient characteristics
RFS

| | HR (95% CI) | p value |
|---|---|---|
| Test (highest vs high/int) | 0.45 (0.25-0.80) | 0.007 |
| Age (<70 vs 70+) | 0.84 (0.46-1.51) | 0.553 |
| T (1 vs 2+) | 2.38 (1.31-4.33) | 0.004 |
| Gender (M vs F) | 0.48 (0.26-0.89) | 0.020 |
| Histology (not adeno vs | 0.96 (0.49-1.89) | 0.916 |
| adeno) | | |

TABLE 40
Multivariate analysis of OS adjusting for
other patient characteristics
OS

| | HR (95% CI) | p value |
|---|---|---|
| Test (highest vs high/int) | 0.63 (0.30-1.35) | 0.238 |
| Age (<70 vs 70+) | 1.33 (0.60-2.97) | 0.483 |
| T (1 vs 2+) | 4.15 (1.97-8.76) | <0.001 |
| Gender (M vs F) | 0.41 (0.18-0.97) | 0.042 |
| Histology (not adeno vs | 1.32 (0.57-3.05) | 0.518 |
| adeno) | | |

TABLE 41
Type of recurrences by test classification:
highest and high/int

| | highest | high/int |
|---|---|---|
| | (N = 56) | (N = 81) |
| Distant (metastatic) | 9 | 5 |
| Locoregional | 10 | 9 |
| New primary | 7 | 7 |

Reproducibility was assessed by comparing the test classifications obtained during development by out-of-bag estimate (on the 62 samples classified as high risk by Classifier A on the development run) with the results obtained from two reruns of the same samples on the ST100. Concordance of the test classifications was 85% and 89%.

3. Second split of the sample set (Split of low risk group from first stratification), Classifier C

This classifier (“Classifier C”) stratifies the low risk group defined by the first classifier (N=177 with 33 recurrences) into two groups with lowest (“lowest”) and intermediate (“low/int”) risk of recurrence.

Eighty-eight patients (50% of the low risk group) were classified to the low/int risk group and the remaining 89 (50%) to the lowest risk group. Fourteen patients in the lowest risk group recurred (16% recurrence rate); nineteen patients in the low/int group recurred (21% recurrence rate). RFS and OS are shown by second split test classification (lowest vs low/int) for patients classified as low risk by the first stratification (Classifier A) in FIGS. 12A and 12B, respectively.

TABLE 42
Time-to-event comparison of the lowest and
low/int subgroups

| | HR (95% CI) | CPH p value | Log-rank p |
|---|---|---|---|
| RFS | 0.61 (0.31-1.21) | 0.159 | 0.155 |
| OS | 0.62 (0.17-2.19) | 0.454 | 0.449 |

TABLE 43
Time-to-event landmarks

| | 1 yr | 2 yr | 3 yr | 5 yr |
|---|---|---|---|---|
| Recurrence-free | 97%/99% | 91%/95% | 82%/90% | 75%/86% |
| (low/int; lowest) | | | | |
| Survival | 100%/100% | 99%/99% | 96%/97% | 92%/93% |
| (low/int; lowest) | | | | |

TABLE 44
Patient characteristics of low risk group by second split test classification

| | | Low/int Risk | Lowest Risk | |
|---|---|---|---|---|
| | | (N = 88) | (N = 89) | |
| | | n (%) | n (%) | P value |
| Gender | Female | 73 (83) | 57 (64) | 0.006 |
| | Male | 15 (17) | 32 (36) | |
| Race | White (including Hispanic) | 84 (95) | 77 (87) | 0.064 |
| | Other | 4 (5) | 12 (13) | |
| Histology | adenocarcinoma | 79 (90) | 80 (90) | >0.999 |
| | other | 9 (10) | 9 (10) | |
| TNM T | 1 | 72 (82) | 71 (80) | 0.849 |
| | 1a | 36 | 35 | (1 vs 2+) |
| | 1b | 15 | 12 | |
| | Not specified | 21 | 24 | |
| | 2 | 13 (15) | 18 (20) | |
| | 2a | 10 | 12 | |
| | 2b | 0 | 0 | |
| | Not specified | 3 | 6 | |
| | 3 | 3 (3) | 0 (0) | |
| Pleural Invasion | yes | 6 (7) | 9 (10) | 0.591 |
| | no | 81 (92) | 80 (90) | (yes vs no) |
| | NA | 1 (1) | 0 (0) | |
| LVI | yes | 10 (11) | 10 (11) | >0.999 |
| | no | 78 (89) | 79 (89) | |
| Size nodules | Median (range) | 1.6 (0.6-13) | 1.6 (0.5-4) | 0.970 |
| Age | Median (range) | 68 (46-87) | 68 (52-89) | 0.312 |

TABLE 45
Types of recurrences by test classification:
lowest and low/int

| | low/int | lowest |
|---|---|---|
| | (N = 88) | (N = 89) |
| Distant (metastatic) | 4 | 3 |
| Locoregional | 9 | 4 |
| New primary | 6 | 7 |

Reproducibility was assessed by comparing the test classifications obtained during development by out-of-bag estimate for samples classified as low risk by Classifier A (N=62) with the results obtained from two additional runs of these samples on the ST100. Concordance of the test classifications was 85% and 89%.

Hierarchical combination of classifiers A, B and C in a testing regime.

As explained previously, and with reference to FIG. 3, combining the three classifiers A, B and C as described above, a four-way classification of patients can be achieved. Spectra are first classified by the “first split” classifier to generate a high risk or low risk classification. Patients with spectra classified as high risk are then classified using the second split classifier for the high risk group to yield a classification of highest or highiint. Patients with spectra classified as low risk are then classified using the second split classifier for the low risk group to yield a classification of lowest or low/int. This is shown schematically in FIG. 3.

For the development sample set in this Section 7 (see above) the patient characteristics by classification label are shown in Table 46.

TABLE 46
Patient characteristics by lowest, low/int, high/int and highest test classifications

| | | Lowest | Low/Int | High/Int | Highest |
|---|---|---|---|---|---|
| | | Risk | Risk | Risk | Risk |
| | | (N = 89) | (N = 88) | (N = 81) | (N = 56) |
| | | n (%) | n (%) | n (%) | n (%) |
| Gender | Female | 57 (64) | 73 (83) | 33 (41) | 34 (61) |
| | Male | 32 (36) | 15 (17) | 48 (59) | 22 (39) |
| Race | White | 77 (87) | 84 (95) | 75 (93) | 50 (89) |
| | Other | 12 (13) | 4 (5) | 5 (6) | 6 (11) |
| | NA | 0 (0) | 0 (0) | 1 (1) | 0 (0) |
| Histology | Adenocarcinoma | 80 (90) | 79 (90) | 64 (79) | 44 (79) |
| | Other | 9 (10) | 9 (10) | 16 (20) | 12 (21) |
| | NA | 0 (0) | 0 (0) | 1 (1) | 0 (0) |
| TNM T | 1 | 71 (80) | 72 (82) | 65 (80) | 37 (66) |
| | 1a | 35 | 36 | 26 | 16 |
| | 1b | 12 | 15 | 19 | 9 |
| | Not specified | 24 | 21 | 20 | 12 |
| | 2 | 18 (20) | 13 (15) | 14 (17) | 18 (32) |
| | 2a | 12 | 10 | 10 | 13 |
| | 2b | 0 | 0 | 0 | 0 |
| | Not specified | 6 | 3 | 4 | 5 |
| | 3 | 0 (0) | 3 (3) | 2 (3) | 1 (2) |
| Pleural | yes | 9 (10) | 6 (7) | 8 (10) | 10 (18) |
| invasion | no | 80 (90) | 81 (92) | 73 (90) | 46 (82) |
| | NA | 0 (0) | 1 (1) | 0 (0) | 0 (0) |
| LVI | yes | 10 (11) | 10 (11) | 4 (5) | 7 (13) |
| | no | 79 (89) | 78 (89) | 77 (95) | 49 (88) |
| Size nodules | Median (range) | 1.6 (0.5-4) | 1.6 (0.6-13) | 1.8 (0.7-3.6) | 1.9 (0.60-5) |
| Age | Median (range) | 68 (52-89) | 68 (46-87) | 73 (49-88) | 71 (46-92) |

Recurrence-free survival and overall survival for the whole development cohort stratified by four-way test classification are shown in FIGS. 13A and 13B, respectively.

TABLE 47
Time-to-event landmarks summary

| Recurrence-free | 1 yr | 2 yr | 3 yr | 5 yr |
|---|---|---|---|---|
| Highest | 85% | 67% | 57% | 47% |
| High/int | 96% | 85% | 81% | 68% |
| Low/int | 97% | 91% | 82% | 75% |
| Lowest | 99% | 95% | 90% | 86% |
| Survival | 1 yr | 2 yr | 3 yr | 5 yr |
| Highest | 98% | 86% | 81% | 69% |
| High/int | 99% | 94% | 91% | 81% |
| Low/int | 100% | 99% | 96% | 92% |
| Lowest | 100% | 99% | 97% | 93% |

TABLE 48
Types of recurrences by test classifications:
lowest, low/int, high/int, and highest

| | highest | high/int | low/int | lowest |
|---|---|---|---|---|
| | (N = 56) | (N = 81) | (N = 88) | (N = 89) |
| Distant (metastatic) | 9 | 5 | 4 | 3 |
| Locoregional | 10 | 9 | 9 | 4 |
| New primary | 7 | 7 | 6 | 7 |

Reproducibility of the 4 way classification was assessed comparing reruns of 124 of the development samples on the ST100 with out-of-bag estimates for the development run of the same samples. Concordance of the classification labels was 80% and 81%.

Alternative hierarchical combination of Classifiers A, B and C: ternary split of the cohort (FIG. 14)

Inspection of FIG. 13A indicates that RFS is similar for the high/int and low/int groups. Hence, a ternary classification of patients can be achieved by combining these two groups into one intermediate group. Spectra are first classified by the “first split” classifier (Classifier A) to generate a high risk or low risk classification. Patients with spectra classified as high risk are then classified using the second split classifier for the high risk group (Classifier B) to yield a classification of highest or intermediate. Patients with spectra classified as low risk are then classified using the second split classifier for the low risk group (Classifier C) to yield a classification of lowest or intermediate. The intermediate classifications produced by classifiers B and C are grouped together and have the same classification label, “intermediate” or the equivalent. This hierarchical combination of classifiers is shown schematically in FIG. 14.

TABLE 4
Patient characteristics by lowest, intermediate and highest test classifications

| | | Lowest | Intermediate | Highest |
|---|---|---|---|---|
| | | Risk | Risk | Risk |
| | | (N = 89) | (N = 169) | (N = 56) |
| | | n (%) | n (%) | n (%) |
| Gender | Female | 57 (64) | 106 (63) | 34 (61) |
| | Male | 32 (36) | 63 (37) | 22 (39) |
| Race | White | 77 (87) | 159 (94) | 50 (89) |
| | Other | 12 (13) | 9 (5) | 6 (11) |
| | NA | 0 (0) | 1 (1) | 0 (0) |
| Histology | Adenocarcinoma | 80 (90) | 143 (85) | 44 (79) |
| | Other | 9 (10) | 25 (15) | 12 (21) |
| | NA | 0 (0) | 1(1) | 0 (0) |
| TNM T | 1 | 71 (80) | 137 (81) | 37 (66) |
| | 1a | 35 | 62 | 16 |
| | 1b | 12 | 34 | 9 |
| | Not specified | 24 | 41 | 12 |
| | 2 | 18 (20) | 27 (16) | 18 (32) |
| | 2a | 12 | 20 | 13 |
| | 2b | 0 | 0 | 0 |
| | Not specified | 6 | 7 | 5 |
| | 3 | 0 (0) | 5 (3) | 1 (2) |
| Pleural | yes | 9 (10) | 14 (8) | 10 (18) |
| invasion | no | 80 (90) | 154 (91) | 46 (82) |
| | NA | 0 (0) | 1 (1) | 0 (0) |
| LVI | yes | 10 (11) | 14 (8) | 7 (13) |
| | no | 79 (89) | 155 (92) | 49 (88) |
| Nodule Size | Median (range) | 1.6 (0.5-4) | 1.7 (0.6-13) | 1.9 (0.60-5) |
| Age | Median (range) | 68 (52-89) | 69(46-88) | 71 (46-92) |

FIGS. 15A and 15B are Kaplan-Meier plots of the time to event outcomes by the ternary test classifications produced by the schema of FIG. 14, namely lowest, intermediate and highest risk.

TABLE 50
Time-to-event comparison of ternary subgroups

| | | | CPH | Log-rank |
|---|---|---|---|---|
| | | HR (95% CI) | p value | p |
| RFS | highest vs intermediate | 0.40 (0.25-0.65) | <0.001 | <0.001 |
| RFS | highest vs lowest | 0.21 (0.11-0.41) | <0.001 | <0.001 |
| RFS | Intermediate vs lowest | 0.53 (0.29-0.97) | 0.041 | 0.038 |
| RFS | highest vs other | 0.33 (0.21-0.52) | <0.001 | <0.001 |
| RFS | other vs lowest | 0.41 (0.23-0.73) | 0.003 | 0.002 |
| OS | highest vs intermediate | 0.43 (0.22-0.84) | 0.013 | 0.011 |
| OS | highest vs lowest | 0.13 (0.04-0.41) | <0.001 | <0.001 |
| OS | Intermediate vs lowest | 0.29 (0.10-0.85) | 0.023 | 0.016 |
| OS | highest vs other | 0.32 (0.17-0.61) | 0.001 | <0.001 |
| OS | other vs lowest | 0.23 (0.08-0.65) | 0.006 | 0.003 |

TABLE 51
Time-to-event landmarks summary

| Recurrence-free | 1 yr | 2 yr | 3 yr | 5 yr |
|---|---|---|---|---|
| highest | 85% | 67% | 57% | 47% |
| intermediate | 96% | 88% | 81% | 72% |
| lowest | 99% | 95% | 90% | 86% |
| Survival | 1 yr | 2 yr | 3 yr | 5 yr |
| highest | 98% | 86% | 81% | 69% |
| intermediate | 99% | 96% | 94% | 87% |
| lowest | 100% | 99% | 97% | 93% |

TABLE 52
Types of recurrences by test classifications:
lowest, intermediate and highest risk

| | highest | intermediate | lowest |
|---|---|---|---|
| | (N = 56) | (N = 169) | (N = 89) |
| Distant (metastatic) | 9 | 9 | 3 |
| locoregional | 10 | 18 | 4 |
| New primary | 7 | 13 | 7 |

TABLE 53
Multivariate analysis of RFS adjusting for other patient
characteristics (ternary classification)
RFS

| | HR (95% CI) | P value |
|---|---|---|
| Test (highest vs lowest) | 0.24 (0.12-0.45) | <0.001 |
| (highest vs intermediate) | 0.45 (0.28-0.74) | 0.002 |
| Age (<70 vs 70+) | 0.95 (0.61-1.49) | 0.826 |
| T (1 vs 2+) | 2.42 (1.53-3.84) | <0.001 |
| Gender (M vs F) | 0.51 (0.32-0.81) | 0.005 |
| Histology (not adeno vs adeno) | 1.21 (0.71-2.08) | 0.490 |

TABLE 54
Multivariate analysis of OS adjusting for other patient
characteristics (ternary classification)
OS

| | HR (95% CI) | P value |
|---|---|---|
| Test (highest vs lowest) | 0.15 (0.05-0.46) | 0.001 |
| (highest vs intermediate) | 0.51 (0.26-1.02) | 0.057 |
| Age (<70 vs 70+) | 1.51 (0.77-2.95) | 0.231 |
| T (1 vs 2+) | 3.33 (1.74-6.35) | <0.001 |
| Gender (M vs F) | 0.50 (0.25-0.99) | 0.047 |
| Histology (not adeno vs adeno) | 1.46 (0.69-3.08) | 0.324 |

TABLE 55
Multivariate analysis of RFS adjusting for other patient
characteristics (highest vs other)
RFS

| | HR (95% CI) | P value |
|---|---|---|
| Test (highest vs other) | 0.37 (0.23-0.59) | <0.001 |
| Age (<70 vs 70+) | 0.97 (0.62-1,52) | 0.891 |
| T (1 vs 2+) | 2.34 (1.47-3.71) | <0.001 |
| Gender (M vs F) | 0.52 (0.33-0.82) | 0.005 |
| Histology (not adeno vs adeno) | 1.25 (0.73-2.14) | 0.415 |

TABLE 56
Multivariate analysis of OS adjusting for other patient characteristics
(highest vs other)
OS

| | HR (95% Cl) | P value |
|---|---|---|
| Test (highest vs other) | 0.37 (0.19-0.72) | 0.004 |
| Age (<70 vs 70+) | 1.49 (0.76-2.92) | 0.242 |
| T (1 vs 2+) | 3.13 (1.63-5.99) | 0.001 |
| Gender (M vs F) | 0.47 (0.24-0.93) | 0.029 |
| Histology (not adeno vs adeno) | 1.46 (0.69-3.07) | 0.320 |

TABLE 57
Multivariate analysis of RFS adjusting for other patient characteristics
(lowest vs other)
RFS

| | HR (95% Cl) | P value |
|---|---|---|
| Test (other vs lowest) | 0.41 (0.23-0.73) | 0.002 |
| Age (<70 vs 70+) | 0.98 (0.63-1.53) | 0.922 |
| T (1 vs 2+) | 2.62 (1.67-4.12) | <0.001 |
| Gender (M vs F) | 0.52 (0.32-0.82) | 0.005 |
| Histology (not adeno vs adeno) | 1.32 (0.78-2.25) | 0.307 |

TABLE 58
Multivariate analysis of OS adjusting for other patient characteristics
(lowest vs other)
OS:

| | HR (95% Cl) | P value |
|---|---|---|
| Test (other vs lowest) | 0.24 (0.08-0.66) | 0.006 |
| Age (<70 vs 70+) | 1.54 (0.79-3.00) | 0.209 |
| T (1 vs 2+) | 3.57 (1.89-6.74) | <0.001 |
| Gender (M vs F) | 0.52 (0.26-1.03) | 0.060 |
| Histology not adeno vs adeno) | 1.63 (0.78-3.39) | 0.190 |

Reproducibility of the ternary classification was assessed comparing reruns of 124 of the development samples on the ST100 with out-of-bag estimates for the development run of the same samples. Concordance of 84% and 86% was observed.

Associations of test classifications with biological processes using PSEA We performed Protein Set Enrichment Analysis to discover the associations between test classifications in the regime of FIG. 14 with biological processes. See the above description and literature cited for more details. The results were as follows.

### 1. High Risk vs Low Risk (Classifier A)

TABLE 59
PSEA p values and FDR for high risk vs low risk phenotypes

| Biological Process | p value | FDR |
|---|---|---|
| Acute inflammatory response | <0.000001 | <0.001 |
| Acute phase response | <0.000001 | <0.001 |
| Complement activation (narrow definition) | <0.000001 | <0.001 |
| Complement activation (broad definition) | 0.000039 | <0.001 |
| Wound healing (narrow definition) | 0.008582 | <0.05 |
| Wound healing (broad definition) | 0.034037 | <0.15 |
| Innate immune response | 0.037454 | <0.15 |
| Immune tolerance | 0.063985 | <0.25 |
| Glycolysis | 0.070078 | <0.25 |
| Cellular component of morphogenesis | 0.128625 | <0.35 |
| Chronic Inflammatory response | 0.137225 | <0.35 |
| Type1 immune response | 0.154531 | <0.35 |
| Epithelial mesenchymal transition | 0.172933 | <0.35 |
| Type2 immune response | 0.198499 | <0.40 |
| Hypoxia | 0.214417 | <0.40 |
| Immune tolerance and suppression | 0.230057 | <0.40 |
| T cell mediated immunity | 0.276113 | <0.45 |
| Interferon type 1 | 0.439324 | <0.65 |
| NK cell mediated immunity | 0.467127 | <0.65 |
| Cytokine production involved in immune response | 0.477872 | <0.65 |
| Angiogenesis | 0.519193 | <0.65 |
| Behavior | 0.671154 | <0.80 |
| Type17 immune response | 0.682384 | <0.80 |
| B cell mediated immunity | 0.782806 | <0.85 |
| Extracellular matrix organization | 0.785794 | <0.85 |
| Interferon Gamma | 0.801015 | <0.85 |

### 2. Highest Risk vs Other

TABLE 60
PSEA p values and FDR for highest risk vs other phenotypes

| Biological Process | p value | FDR |
|---|---|---|
| Acute phase response | <0.000001 | <0.001 |
| Complement activation (narrow definition) | <0.000001 | <0.001 |
| Complement activation (broad definition) | <0.000001 | <0.001 |
| Acute inflammatory response | 0.000099 | <0.001 |
| Wound healing (narrow definition) | 0.000590 | <0.005 |
| Immune tolerance | 0.001163 | <0.01 |
| Immune tolerance and suppression | 0.006420 | <0.05 |
| Chronic Inflammatory response | 0.041551 | <0.15 |
| B cell mediated immunity | 0.061018 | <0.20 |
| Wound healing (broad definition) | 0.069594 | <0.20 |
| NK cell mediated immunity | 0.086320 | <0.25 |
| Innate immune response | 0.121993 | <0.30 |
| Interferon type 1 | 0.148747 | <0.30 |
| Cytokine production involved in | 0.192156 | <0.35 |
| immune response | | |
| Type1 immune response | 0.196954 | <0.35 |
| Cellular component of morphogenesis | 0.359505 | <0.60 |
| Glycolysis | 0.374186 | <0.60 |
| Epithelial mesenchymal transition | 0.476837 | <0.70 |
| Extracellular matrix organization | 0.484809 | <0.70 |
| Interferon Gamma | 0.559108 | <0.75 |
| Hypoxia | 0.590999 | <0.75 |
| Behavior | 0.660899 | <0.80 |
| Type2 immune response | 0.802948 | <0.85 |
| T cell mediated immunity | 0.824178 | <0.85 |
| Angiogenesis | 0.845749 | <0.85 |
| Type17 immune response | 0.849596 | <0.85 |

### 3. Lowest Risk vs Other

TABLE 61
PSEA p values and FDR for lowest risk vs other phenotypes

| Biological Process | p value | FDR |
|---|---|---|
| Acute phase response | <0.000001 | <0.001 |
| Acute inflammatory response | 0.000039 | <0.001 |
| Complement activation (narrow definition) | 0.000039 | <0.001 |
| Complement activation (broad definition) | 0.000176 | <0.005 |
| Wound healing (narrow definition) | 0.004007 | <0.05 |
| Wound healing (broad definition) | 0.004651 | <0.05 |
| Cellular component of morphogenesis | 0.026420 | <0.10 |
| Angiogenesis | 0.081998 | <0.30 |
| Immune tolerance | 0.087838 | <0.30 |
| Chronic Inflammatory response | 0.105875 | <0.30 |
| Immune tolerance and suppression | 0.143916 | <0.35 |
| Glycolysis | 0.163333 | <0.40 |
| Cytokine production involved in | 0.207082 | <0.45 |
| immune response | | |
| Hypoxia | 0.317116 | <0.60 |
| Type1 immune response | 0.325730 | <0.60 |
| Type2 immune response | 0.393428 | <0.65 |
| Interferon Gamma | 0.439010 | <0.70 |
| Type17 immune response | 0.474887 | <0.70 |
| T cell mediated immunity | 0.502194 | <0.70 |
| B cell mediated immunity | 0.580645 | <0.80 |
| Innate immune response | 0.719421 | <0.90 |
| Interferon type 1 | 0.748498 | <0.90 |
| Extracellular matrix organization | 0.766493 | <0.90 |
| NK cell mediated immunity | 0.791051 | <0.90 |
| Behavior | 0.909113 | <0.95 |
| Epithelial mesenchymal transition | 0.983263 | <1.00 |

### 4. Highest Risk vs Lowest Risk

TABLE 62
PSEA p values and FDR for low/int risk vs lowest risk phenotypes

| Biological Process | p value | FDR |
|---|---|---|
| Acute phase response | 0.000020 | <0.001 |
| Complement activation (narrow definition) | 0.000356 | <0.005 |
| Acute inflammatory response | 0.002683 | <0.05 |
| Complement activation (broad definition) | 0.003986 | <0.05 |
| Wound healing (narrow definition) | 0.048576 | <0.30 |
| Immune tolerance | 0.086054 | <0.35 |
| Angiogenesis | 0.091256 | <0.35 |
| Innate immune response | 0.182520 | <0.60 |
| Cellular component of morphogenesis | 0.209831 | <0.60 |
| Wound healing (broad definition) | 0.222516 | <0.60 |
| Chronic Inflammatory response | 0.254650 | <0.65 |
| Cytokine production involved in immune response | 0.304471 | <0.70 |
| Glycolysis | 0.422005 | <0.75 |
| Interferon Gamma | 0.494367 | <0.75 |
| Immune tolerance and suppression | 0.503638 | <0.75 |
| Type17 immune response | 0.521282 | <0.75 |
| Behavior | 0.540987 | <0.75 |
| NK cell mediated immunity | 0.542131 | <0.75 |
| Type1 immune response | 0.543990 | <0.75 |
| Extracellular matrix organization | 0.639250 | <0.80 |
| B cell mediated immunity | 0.662902 | <0.80 |
| Hypoxia | 0.684290 | <0.80 |
| Interferon type 1 | 0.684530 | <0.80 |
| Epithelial mesenchymal transition | 0.831538 | <0.95 |
| Type2 immune response | 0.983664 | <1.00 |
| T cell mediated immunity | 0.984818 | <1.00 |

Conclusions of Redevelopment of Risk of Recurrence Test (Section 7)

We were able to create a suite of three classifiers (A, B and C) stratifying patients with early stage lung cancer by risk of recurrence. Eighteen percent of patients were assigned to the highest risk group, 54% to the intermediate risk group (26% to the high/intermediate risk group, 28% to the low/intermediate risk group) and 28% to the lowest risk group. The percentage of patients recurrence-free at two years varied from 67% in the highest risk group to 95% in the lowest risk group; the percentage of patients alive at five years was 69% in the highest risk group and 93% in the lowest risk group. RFS and OS were significantly different between highest risk, intermediate risk and lowest risk classifications and they remained predictive of RFS and OS (trend for intermediate vs highest risk for OS) in multivariate analysis, adjusting for other prognostic factors. It is noteworthy that the tests were able to stratify all three kinds of recurrence: distant, locoregional and new primary, although performance was best for distant and locoregional recurrences.

Set enrichment analysis indicated that test classifications were associated with acute phase response, complement activation, acute inflammatory response, and wound healing. Immune tolerance could also be potentially relevant. These observations, together with our experience showing the relevance of complement, wound healing, acute phase response and acute inflammatory response in metastatic cancer treated with immunotherapies and the fact that the classifiers are able to stratify risk of new primary lesions, could indicate that the test is accessing information on the host's immune response to cancer.

Reproducibility of the test classifications was good, with reproducibility of around 85% for the ternary classification of highest, intermediate and lowest risk.

While the ternary test appeared to work well on plasma (i.e. produced concordant classifications between serum and plasma within the inherent reproducibility of the serum test itself), the first split of the dataset (binary classification) did not. Further investigations should be undertaken to assess whether the apparent correction to concordance on moving from 4-way to ternary classification is reliable if the ternary test is to run on plasma samples.

Analysis of test performance in the large subgroup of patients with adenocarcinoma demonstrated similar performance to that in the whole cohort.

Section 8: Development and Use of a Classifier Developed from Samples Obtained Post-Surgery

We had post-surgery samples collected between 30 and 120 days after surgery in addition to pre-surgery samples from 114 patients. We found that applying the above-described redeveloped risk of recurrence test. developed on 300+ patients (described in Section 7) to these post-surgery samples was not very useful. However, we did discover that if we excluded the patients we had identified as at highest risk of recurrence from their pre-surgery sample, we could make a test using post-surgery samples that allowed a better stratification of these patients into intermediate and lowest risk groups.

In practical terms, one could implement the test (or classifier) described in this section after surgery, in addition to performing a test from a blood-based sample prior to surgery. In particular, one would test a patient pre-surgery, using the test of Section 7 (e.g., a ternary classification routine as described in that section). If the pre-surgery sample is classified as highest risk, that test result could inform and guide their treatment. For example, it could lead to adjuvant chemotherapy, or perhaps immunotherapy if such treatment is approved in the future, or more intensive follow up with the patient. If the pre-surgery patient is classified as lowest or intermediate risk, we could obtain a post-surgery serum sample and generate an improved stratification based on that, using the classifier developed as described in this section.

As the classifier developed in this section only had samples collected 30-120 days post-surgery, we do not presently know if that is an optimal timeframe in which to collect a second sample. In one possible strategy, stratification could be improved by collecting a series of post-surgery samples (e.g. at 6 months, 9 months, 1 year post-surgery) and conducting the test described in this section on each of such samples.

Our observation we have made is that the serum proteome changes from pre-surgery to post-surgery, and the post-surgery proteome contains information that allows us to improve our recurrence risk stratification. We have conducted analysis of PSEA scores, which support the realization that there are significant changes between pre- and post-surgery sampling,

A post-surgery classifier was developed by training on the post-surgery feature values derived from the first spectral acquisition using instrument “ST100”, as mentioned earlier. Patients whose pre-surgery samples were classified as highest risk by the pre-surgery classifier were excluded, leaving 95 post-surgery samples for classifier development. The resulting classifier stratifies patients into a group with higher risk of recurrence (class label “G1”) and lower risk (class label “G2”). In this section, the highest-risk pre-surgery patients are shown alongside the plots for the patients having class label G1 and G2 for purposes of comparison despite such the fact that samples from such patients were not used in the post-surgery classifier development.

Details of Classifier Development

A classifier was developed using the procedure shown in FIG. 2, as described in detail previously. The development samples were initially assigned a training class label based on RFS. Samples with RFS less than the median value were assigned to G1 and samples with RFS greater than the median value were assigned to G2, regardless of outcome. An iterative label-flip approach was used to generate training class labels consistent with the labels that the classifier produced. The atomic classifiers were k-nearest-neighbor classifiers with k=9. Atomic classifiers corresponding to all features and pairs of features were created and then filtered so that only atomic classifiers resulting in an RFS hazard ratio between classifications of at least 2.5 were used. Master classifiers were generated using dropout logistic regression combination with 10 atomic classifiers left in for each of 100,000 dropout iterations,

Results

After classifier development, the matched samples were classified using the post-surgery classifier, using out-of-bag classifications, with those patients designated highest risk based on their pre-surgery ST100 classification excluded. Of the 114 matched samples, 24 (21%) were classified as highest risk by the pre-surgery classifier, 49 (43%) were classified as G1, and 41 (36%) were classified as G2 (Table 63). Of the 22 recurrences in the matched sample cohort, of which eight belonged to the highest-risk group (33% recurrence rate in this group), 12 were assigned to G1 (24% recurrence rate), and two to G2 (5% recurrence rate),

TABLE 63
Post-surgery classifications of post-surgery samples

| | N (%) |
|---|---|
| Pre-surgery highest risk | 24 (21) |
| G1 (higher risk) | 49 (43) |
| G2 (lower risk) | 41 (36) |

The concordance between the post-surgery classifier (using the post-surgery samples) and the original pre-surgery ROR classifier (using the pre-surgery samples) is shown in Table 64 for patients not classified as at highest risk of recurrence from their pre-surgery sample. Thirteen of the patients whose pre-surgery samples were classified as low risk were classified as G1 (higher risk) post-surgery, of which two patients had recurrences. Twelve patients were classified as intermediate risk pre-surgery and as G2 (lower risk) after surgery, of which no patients recurred.

TABLE 64
Concordance of post-surgery classifications
and original pre-surgery ROR classifications

| | | Post-surgery classifier | | |
|---|---|---|---|---|
| | | G1 | G2 | Total |
| Pre-surgery | Intermediate | 36 | 12 | 48 |
| classifier | Low | 13 | 29 | 42 |
| | Total | 49 | 41 | 90 |

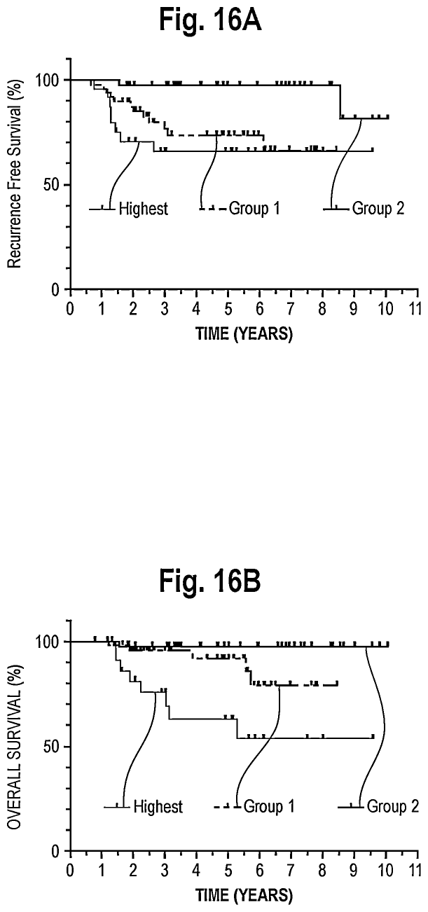

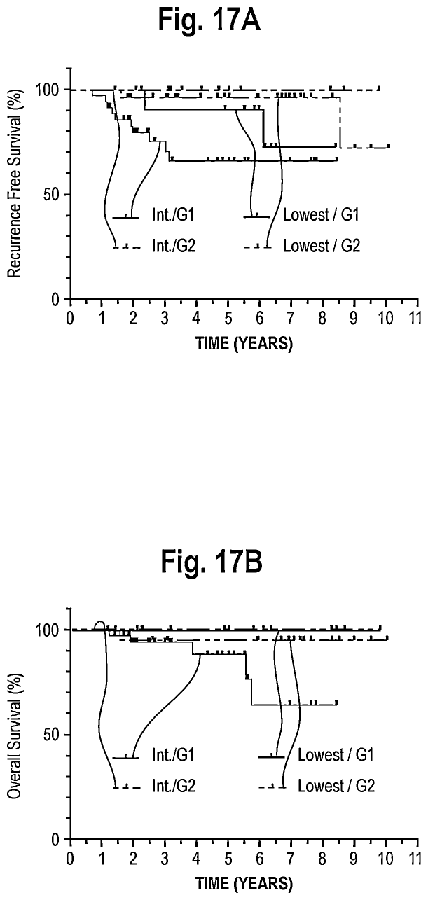

Recurrence-free survival is shown by test classification in FIG. 16A and 16B, An RFS plot split on pre-surgery classification (Int./Low) as well as post-surgery classification (G1/G2) is shown in FIG. 17A and 17B for samples not classified as highest risk by the pre-surgery classifier. In FIG. 17B the horizontal line at the top is Int/G2 and Lowest/G1 (the lines overlap).
Cox proportional hazard ratios and p values comparing G1 vs G2 are shown in Table 65.

TABLE 65
Hazard ratios and p-values for the comparison of time-to-event
outcomes between G1 and G2

| | HR (95% Cl) | p-value |
|---|---|---|
| RFS | 0.08 (0.01-0.60) | 0.014 |
| OS | 0.19 (0.02-1.61) | 0.127 |

Some key time-to-event landmarks are summarized in Table.

TABLE 66
Time-to-event landmarks by post-surgery
test classification

| | 1 year | 2 years | 3 years | 5 years |
|---|---|---|---|---|
| RFS (%) | | | | |
| Highest | 96 | 71 | 66 | 66 |
| Group1 | 98 | 85 | 77 | 74 |
| Group2 | 100 | 98 | 98 | 98 |
| OS (%) | | | | |
| Highest | 100 | 81 | 75 | 63 |
| Group1 | 100 | 96 | 96 | 92 |
| Group2 | 100 | 98 | 98 | 98 |

Table 67 shows patient characteristics by test classification.

TABLE 67
Patient characteristics by post-surgery test classification

| | | Group 1 | Group 2 | P value |
|---|---|---|---|---|
| | | (N = 49) | (N = 41) | (G1 vs |
| | | n (%) | n (%) | G2) |
| Gender | Female | 29 (59) | 29 (71) | 0.277 |
| | Male | 20 (41) | 12 (29) | |
| Race | White (including | 43 (88) | 35 (85) | 0.748 |
| | Hispanic) | | | |
| | Other | 5 (10) | 6 (14) | (White vs |
| | NA | 1 (2) | 0 (0) | Other) |
| Histology | adenocarcinoma | 43 (88) | 39 (95) | 0.283 |
| | other | 6 (12) | 2 (5) | |
| TNM T | 1 | 39 (80) | 36 (88) | 0.398 |
| | 1a | 17 | 19 | (1 vs 2+) |
| | 1b | 9 | 6 | |
| | Not specified | 13 | 11 | |
| | 2 | 8 (16) | 4 (10) | |
| | 2a | 7 | 2 | |
| | 2b | 0 | 0 | |
| | Not specified | 1 | 2 | |
| | 3 | 2 (4) | 1 (2) | |
| Pleural | yes | 4 (8) | 1 (2) | 0.371 |
| Invasion | no | 45 (92) | 40 (98) | |
| LVI | yes | 2 (4) | 3 (7) | 0.656 |
| | no | 47 (96) | 38 (93) | |
| # nodules | 1 | 46 (94) | 41 (100) | 0.248 |
| | Median size | 1.6 (0.7-8) | 1.8 (0.5-13) | (1 vs 2) |
| | (cm) (range) | | | |
| | 2 | 3 (6) | 0 (0) | |
| | Median total | 4.3 (3.7-4.5) | | |
| | size (cm) (range) | | | |
| Age | Median (range) | 72 (46-89) | 69 (54-83) | |

Table 68 shows the ability of the test to predict recurrence-free survival when adjusted for other patient characteristics. Among the recurrences, G1 and G2 both contained roughly equal proportions of locoregional recurrences and new primaries, although the total number of recurrences in G2 is very small, making comparisons difficult. Table 69 shows the types of recurrences by test classification.

TABLE 68
Multivariate analysis of RFS and OS adjusting for other patient characteristics

| | HR (95% Cl) | p-value |
|---|---|---|
| RFS | | |
| Test (G1 vs G2) | 0.08 (0.01-0.64) | 0.017 |
| Gender (M vs F) | 0.20 (0.05-0.76) | 0.018 |
| TNM T stage (1 vs 2+) | 4.59 (1.45-14.48) | 0.009 |
| Age (<70 vs 70+) | 0.42 (0.12-1.47) | 0.175 |
| Histology (adeno vs other) | 0.86 (0.21-3.66) | 0.858 |
| OS | | |
| Test (G1 vs G2) | 0.22 (0.02-1.95) | 0.172 |
| Gender (M vs F) | 0.06 (0.01-0.66) | 0.022 |
| TNM T stage (1 vs 2+) | 3.51 (0.49-25.27) | 0.213 |
| Age (<70 vs 70+) | 0.43 (0.07-2.57) | 0.357 |
| Histology (adeno vs other) | 0.60 (0.05-7.86) | 0.704 |

TABLE 69
Types of recurrence by test classification: Pre-surgery
highest, G1, and G2

| | Pre-surgery | Post-surgery | Post-surgery |
|---|---|---|---|
| | Highest (N = 24) | G1 (N = 49) | G2 (N = 41) |
| Distant (metastatic) | 3 | 1 | 0 |
| Locoregional | 3 | 5 | 1 |
| New primary | 2 | 6 | 1 |

Reproducibility was assessed by comparing the test classifications obtained during development by out-of-bag estimate with the results obtained from a rerun of the same samples on the ST100, Eighty-nine out of 90 samples (99%) received the same classification for both runs.

Conclusions

A test developed using post-surgery samples, collected from patients not classified as at highest risk of recurrence based on pre-surgery samples, was able to effectively stratify these patients into two groups (G1 and G2) with worse and better RFS and OS, respectively. This stratification of these patients appeared to be better than that obtained from pre-surgery samples and the risk of recurrence test described in Section 7. As the post-surgery test can only be effectively applied to patients not classified as at highest risk based on pre-surgery samples, it would be necessary to have tested a patient's pre-surgery sample to provide an improved prognostication of likelihood of recurrence after surgery,

This result indicates the presence of outcome-associated differences in the serum proteome between samples collected before and after surgery. This observation was confirmed by comparing the PSEA scores before and after surgery, the details of which are omitted for the sake of brevity.

Thus, we contemplate a testing methodology as follows.
1. Obtain a pre-surgery blood-based sample from a NSCLC patient, perform mass spectrometry on the sample and obtain the integrated intensity values of the features listed in Appendix A, and then classify the mass spectrum of the sample in accordance with the testing procedure of either Section 4 or Section 7 (and such a test, using one or more classifiers described in these sections, could be configured as a binary classifier, a ternary classifier, or a four-way classifier as described in these sections).
2. If the sample is not classified as having a high or highest risk of recurrence in accordance with the classification produced in step (1), obtain a further blood-based sample from the patient after surgery and conduct mass spectrometry on the blood-based sample including obtaining integrated intensity values of the features listed in Appendix A.
3. Classify the mass spectrum of the sample obtained in 2. in accordance with the testing procedure of this Section. The class label will be reported as either G1 or the equivalent and G2 or the equivalent, with G2-labeled patients predicted to do better in terms of RFS and OS, as compared to patients with the class label G1, as indicated by the plots of FIGS. 16 and 17.
4. Steps 2 and 3 could be repeated over time, in order to obtain longitudinal classifications of the sample. If and when the samples change class label from G2 to G1 then the patient could be guided to more aggressive treatment, e.g., adjuvant chemotherapy, immunotherapy, radiation therapy or more close follow-up.

Section 9, Further Considerations

Practical implementations of the test of this document could take several forms.

In one embodiment, a method for performing a risk assessment of recurrence of cancer in an early stage non-small-cell lung cancer patient includes the steps of:

(a) performing mass spectrometry on a blood-based sample obtained from the patient and obtaining mass spectrometry data, and

(b) in a computing machine, performing a hierarchical classification procedure on the mass spectrometry data wherein the computing machine implements a hierarchical classifier schema including a first classifier (Classifier A) producing a class label in the form of high risk or low risk or the equivalent (See FIGS. 3, 14) and if the Classifier A produces the high risk label the sample is classified by a second classifier (Classifier B) generating a classification label of highest risk or high/intermediate risk or the equivalent, wherein if Classifier B produces the label of highest risk or the equivalent the patient is predicted to have a high risk of recurrence of the cancer following surgery. For example, in this situation, the patient could be guided to more aggressive treatments for the cancer, such as by suggesting or prescribing adjuvant chemotherapy or radiation treatment,

Alternatively, the test could be performed in accordance with a method in which the computing machine implements a hierarchical classifier schema including a third classifier (Classifier C), see FIGS. 3 and 14, wherein if the classifier A produces a “low risk” (or not “high risk, or the equivalent) classification label the sample is classified by the third classifier C and wherein classifier C produces a class label of lowest risk or low/intermediate risk or the equivalent. In this scenario, the lowest risk class label indicates that the patient providing the sample has a relatively low risk of recurrence of the cancer following surgery.

As described in conjunction with FIGS. 3 and 14, the tests described above could also be implemented in either a four-way or three way (ternary) hierarchical classification approach, such classifiers B and C produce intermediate labels that are neither highest risk nor lowest risk. These intermediate labels could be combined into a general “intermediate” classification label, or the equivalent, as shown in FIG. 14.

As an alternative, the test could be conducted in binary classification procedure using just Classifier A to produce High Risk or Low Risk classification labels (or the equivalent).ln this regard, a method for performing a risk assessment of recurrence of cancer in an early stage non-small-cell lung cancer patient includes the steps of: performing mass spectrometry on a blood-based sample obtained from the patient prior to surgery to treat the cancer and obtaining mass spectrometry data, and in a computing machine, performing a binary classification procedure on the mass spectrometry data wherein the computing machine implements a first classifier (Classifier A) producing a class label in the form of high risk or low risk or the equivalent, wherein if the class label of is high risk or the equivalent the patient is predicted to have a high risk of recurrence of the cancer following surgery.

In the above methods, in one embodiment the computing machine stores a reference set of mass spectrometry data obtained from blood-based samples obtained from a multitude of early stage non-small-cell cancer patients for use in classification of the mass spectrum of the sample, and wherein the mass spectrometry data includes feature values for features listed in Appendix A.

As another example of how the present disclosure can be practiced, a programmed computer is provided with machine-readable code and memory storing parameters for at least Classifier A, and optionally Classifiers B and Classifier C (and code for implementing an associated hierarchical classification schema, shown in FIG. 3 or 14) for making a prediction of the risk of recurrence of cancer in an early stage non-small-cell lung cancer patient. The programmed computer includes a processing unit and a memory storing code and classifier parameters such that the computer is configured as a hierarchical classifier that predicts if a patient is at a high risk of recurrence (from Classifier A or by combining Classifiers A and B) and wherein the memory further storing a reference set of mass spectral data from a multitude of early stage non-small cell lung cancer patients including feature values of the features listed in Appendix A. In one possible configuration the programmed computer includes parameters defining classifiers A, B and C and a hierarchical combination schema as shown in either FIG. 3 or 14 and described above.

In one possible implementation, the classifiers A, B and C are generated from performing the method of FIG. 2 on a development set of samples and take the form of a combination of a multitude of master classifiers each developed from a different separation of the development sample sets into training and test sets.

It will be appreciated that the terms assigned to class labels, such as “high risk” or “highest” are descriptive and offered by way of example but not limitation, and of course other labels could be chosen, such as “good” “bad”, “1”, “2”, “G1” or Group 1, “G2”, etc. The particular nomenclature used in practice is not particularly important.

As noted, in one possible configuration just Classifier A is used to stratify the patient into high and low risk groups. The cases in which one might use just Classifier A for high/low risk and not prefer to define a “highest” risk group (using Classifier B) would be:

1. A scenario where the highest risk identification (produced by Classifier B) does not validate well. Usually our tests validate well, but in this risk of recurrence setting we are dealing with relatively small numbers of recurrers and this increases the risk of not generalizing well. This can be due to some overfitting, misjudging performance on small development set, or not having a population-representative set to train with.
2. That this option would extend better to other indications. As this “first split” of the dataset looks less deeply into the proteome and specifics of the training set, it might be more portable to other indications in terms of moving to stage II NSCLC, other lung cancer or possibly other early stage cancers.

The appended claims are offered as further descriptions of the disclosed inventions.

APPENDIX A
List of Feature Definitions
The features marked by an asterisk (*) were removed from the
final feature table, and used only for batch correction

| | Center | Right |
|---|---|---|
| Left m/z | m/z | m/z |
| 3071.22 | 3085.19 | 3099.16 |
| 3099.64 | 3111.21 | 3122.77 |
| 3125.22 | 3137.00 | 3148.78 |
| 3149.02 | 3156.94 | 3164.86 |
| 3165.70 | 3177.13 | 3188.57 |
| 3189.67 | 3198.85 | 3208.03 |
| 3208.33 | 3216.82 | 3225.30 |
| 3231.00 | 3243.53 | 3256.07 |
| 3256.90 | 3267.00 | 3277.10 |
| 3305.44 | 3314.98 | 3324.51 |
| 3353.79 | 3366.37 | 3378.94 |
| 3384.77 | 3396.02 | 3407.27 |
| 3410.04 | 3422.21 | 3434.37 |
| 3434.51 | 3443.90 | 3453.30 |
| 3454.74 | 3466.38 | 3478.02 |
| 3540.72 | 3555.53 | 3570.35 |
| 3583.14 | 3593.09 | 3603.05 |
| 3667.06 | 3681.88 | 3696.70 |
| 3697.35 | 3705.33 | 3713.31 |
| 3747.09 | 3755.81 | 3764.52 |
| 3766.63 | 3776.40 | 3786.17 |
| 3811.87 | 3821.31 | 3830.74 |
| 3832.00 | 3841.64 | 3851.28 |
| 3860.09 | 3867.51 | 3874.93 |
| 3877.78 | 3888.14 | 3898.49 |
| 3899.28 | 3907.43 | 3915.58 |
| 3915.70 | 3927.75 | 3939.80 |
| 3943.30 | 3952.26 | 3961.21 |
| 3999.11 | 4011.41 | 4023.71 |
| 4023.91 | 4031.43 | 4038.96 |
| 4039.25 | 4051.40 | 4063.54 |
| 4080.14 | 4094.83 | 4109.53 |
| 4112.17 | 4119.37 | 4126.57 |
| 4127.25 | 4133.39 | 4139.52 |
| 4198.95 | 4210.81 | 4222.68 |
| 4258.83 | 4266.50 | 4274.18 |
| 4276.79 | 4289.24 | 4301.69 |
| 4332.05 | 4341.63 | 4351.22 |
| 4351.40 | 4359.83 | 4368.27 |
| 4372.40 | 4381.03 | 4389.66 |
| 4397.29 | 4407.28 | 4417.26 |
| 4427.40 | 4433.22 | 4439.04 |
| 4439.75 | 4443.96 | 4448.18 |
| 4449.38 | 4461.23 | 4473.07 |
| 4502.53 | 4508.75 | 4514.98 |
| 4553.30 | 4565.57 | 4577.84 |
| 4580.87 | 4586.84 | 4592.81 |
| 4593.22 | 4599.59 | 4605.96 |
| 4618.51 | 4625.99 | 4633.46 |
| 4633.75 | 4642.42 | 4651.09 |
| 4667.54 | 4679.99 | 4692.43 |
| 4698.76 | 4713.31 | 4727.86 |
| 4747.49 | 4755.82 | 4764.15 |
| 4770.57 | 4776.34 | 4782.12 |
| 4782.62 | 4790.85 | 4799.08 |
| 4807.16 | 4819.05 | 4830.95 |
| 4845.90 | 4857.70 | 4869.49 |
| 4885.05 | 4893.33 | 4901.61 |
| 4910.19 | 4919.12 | 4928.06 |
| 4928.26 | 4938.24 | 4948.23 |
| 4949.44 | 4964.30 | 4979.15 |
| 4989.38 | 5000.07 | 5010.76 |
| 5012.17 | 5020.40 | 5028.63 |
| 5033.64 | 5041.17 | 5048.71 |
| 5048.95 | 5054.98 | 5061.00 |
| 5061.10 | 5070.88 | 5080.67 |
| 5093.87 | 5106.47 | 5119.06 |
| 5120.38 | 5127.97 | 5135.56 |
| 5162.99 | 5185.80 | 5208.61 |
| 5209.58 | 5224.44 | 5239.30 |
| 5240.40 | 5251.05 | 5261.69 |
| 5274.04 | 5288.09 | 5302.14 |
| 5351.59 | 5362.36 | 5373.12 |
| 5396.97 | 5404.07 | 5411.16 |
| 5411.52 | 5418.07 | 5424.63 |
| 5424.89 | 5431.54 | 5438.18 |
| 5442.72 | 5449.54 | 5456.36 |
| 5512.62 | 5520.50 | 5528.38 |
| 5540.44 | 5552.25 | 5564.06 |
| 5564.15 | 5573.62 | 5583.09 |
| 5685.16 | 5693.39 | 5701.62 |
| 5701.82 | 5708.30 | 5714.77 |
| 5714.97 | 5720.49 | 5726.01 |
| 5726.03 | 5734.42 | 5742.81 |
| 5743.56 | 5750.24 | 5756.93 |
| 5757.29 | 5764.16 | 5771.03 |
| 5771.12 | 5778.62 | 5786.11 |
| 5786.29 | 5794.96 | 5803.62 |
| 5803.89 | 5810.08 | 5816.27 |
| 5816.42 | 5822.76 | 5829.11 |
| 5832.02 | 5840.46 | 5848.89 |
| 5850.08 | 5863.91 | 5877.73 |
| 5879.59 | 5888.74 | 5897.90 |
| 5898.07 | 5909.77 | 5921.47 |
| 5922.62 | 5934.73 | 5946.84 |
| 5949.41 | 5963.90 | 5978.40 |
| 5978.83 | 5987.76 | 5996.69 |
| 5998.01 | 6008.58 | 6019.14 |
| 6020.13 | 6028.93 | 6037.72 |
| 6054.61 | 6061.94 | 6069.27 |
| 6069.47 | 6082.86 | 6096.26 |
| 6099.57 | 6109.12 | 6118.68 |
| 6134.48 | 6148.68 | 6162.88 |
| 6165.75 | 6175.04 | 6184.34 |
| 6186.65 | 6194.45 | 6202.25 |
| 6202.33 | 6209.35 | 6216.38 |
| 6216.68 | 6224.86 | 6233.04 |
| 6275.16 | 6284.15 | 6293.14 |
| 6293.16 | 6301.49 | 6309.82 |
| 6322.27 | 6331.46 | 6340.64 |
| 6378.77 | 6393.09 | 6407.42 |
| 6409.41 | 6479.04 | 6548.68 |
| 6553.89 | 6564.68 | 6575.47 |
| 6575.85 | 6589.26 | 6602.67 |
| 6604.74 | 6675.06 | 6745.39 |
| 6779.07 | 6798.12 | 6817.17 |
| 6825.83 | 6837.67 | 6849.52 |
| 6849.89 | 6859.44 | 6868.99 |
| 6869.08 | 6878.92 | 6888.75 |
| 6889.03 | 6896.99 | 6904.95 |
| 6911.60 | 6920.97 | 6930.34 |
| 6930.88 | 6939.55 | 6948.22 |
| 6948.87 | 6956.18 | 6963.49 |
| 6963.58 | 6971.11 | 6978.64 |
| 6979.01 | 6995.27 | 7011.52 |
| 7011.77 | 7019.83 | 7027.88 |
| 7029.37 | 7033.60 | 7037.84 |
| 7037.91 | 7046.82 | 7055.73 |
| 7055.81 | 7060.15 | 7064.50 |
| 7065.49 | 7072.90 | 7080.31 |
| 7118.24 | 7143.95 | 7169.66 |
| 7178.66 | 7189.32 | 7199.97 |
| 7234.04 | 7243.67 | 7253.30 |
| 7279.59 | 7292.85 | 7306.11 |
| 7309.51 | 7318.12 | 7326.73 |
| 7327.41 | 7332.74 | 7338.06 |
| 7375.19 | 7390.07 | 7404.95 |
| 7406.19 | 7448.51 | 7490.84 |
| *7553.58 | 7566.50 | 7579.42 |
| *7659.19 | 7672.34 | 7685.48 |
| 7731.02 | 7736.79 | 7742.56 |
| 7742.75 | 7751.34 | 7759.93 |
| 7760.24 | 7767.77 | 7775.30 |
| 7776.52 | 7788.92 | 7801.31 |
| 7803.18 | 7820.32 | 7837.46 |
| *7924.01 | 7937.38 | 7950.75 |
| 7984.80 | 7994.91 | 8005.01 |
| 8006.66 | 8018.69 | 8030.72 |
| *8030.75 | 8041.85 | 8052.96 |
| 8131.01 | 8153.05 | 8175.09 |
| 8192.54 | 8215.68 | 8238.82 |
| 8306.66 | 8314.70 | 8322.74 |
| 8353.19 | 8366.02 | 8378.85 |
| 8401.71 | 8411.17 | 8420.63 |
| 8420.71 | 8428.79 | 8436.87 |
| 8466.84 | 8474.84 | 8482.84 |
| 8483.32 | 8489.05 | 8494.77 |
| 8516.01 | 8528.96 | 8541.91 |
| 8555.29 | 8565.12 | 8574.94 |
| 8575.31 | 8592.03 | 8608.74 |
| 8650.35 | 8659.11 | 8667.86 |
| 8754.04 | 8766.76 | 8779.48 |
| 8799.09 | 8820.53 | 8841.97 |
| 8860.56 | 8871.76 | 8882.96 |
| 8882.98 | 8891.91 | 8900.84 |
| 8904.09 | 8925.16 | 8946.24 |
| 8954.36 | 8961.34 | 8968.33 |
| 8968.81 | 8978.23 | 8987.65 |
| 8988.02 | 8998.68 | 9009.33 |
| 9010.43 | 9019.53 | 9028.62 |
| 9028.78 | 9037.31 | 9045.84 |
| 9066.55 | 9077.91 | 9089.26 |
| 9089.32 | 9096.91 | 9104.51 |
| 9112.47 | 9133.46 | 9154.45 |
| 9196.31 | 9207.88 | 9219.45 |
| 9234.27 | 9243.94 | 9253.60 |
| 9254.17 | 9263.30 | 9272.44 |
| 9272.68 | 9289.41 | 9306.14 |
| 9308.35 | 9319.83 | 9331.31 |
| 9341.10 | 9374.82 | 9408.53 |
| 9411.21 | 9454.03 | 9496.84 |
| 9560.23 | 9585.25 | 9610.26 |
| 9613.48 | 9626.56 | 9639.65 |
| 9639.94 | 9647.57 | 9655.20 |
| 9688.55 | 9723.57 | 9758.58 |
| 9903.45 | 9934.33 | 9965.21 |
| 10128.04 | 10139.87 | 10151.71 |
| 10152.46 | 10161.84 | 10171.22 |
| 10171.98 | 10184.57 | 10197.16 |
| 10197.54 | 10211.07 | 10224.60 |
| 10249.52 | 10262.23 | 10274.94 |
| 10295.62 | 10305.69 | 10315.75 |
| 10328.34 | 10350.14 | 10371.93 |
| 10435.64 | 10450.45 | 10465.26 |
| 10465.61 | 10482.62 | 10499.63 |
| 10518.75 | 10564.38 | 10610.01 |
| 10615.18 | 10638.37 | 10661.56 |
| 10711.79 | 10737.82 | 10763.85 |
| 10764.79 | 10775.15 | 10785.51 |
| 10828.47 | 10847.99 | 10867.50 |
| 10951.44 | 10963.37 | 10975.30 |
| 11028.77 | 11056.40 | 11084.03 |
| 11090.89 | 11107.43 | 11123.96 |
| 11132.45 | 11152.43 | 11172.40 |
| 11285.82 | 11305.10 | 11324.39 |
| 11378.42 | 11392.26 | 11406.11 |
| 11428.16 | 11442.74 | 11457.32 |
| 11468.24 | 11485.30 | 11502.35 |
| 11513.71 | 11530.99 | 11548.26 |
| 11567.26 | 11584.42 | 11601.59 |
| 11611.34 | 11634.82 | 11658.30 |
| 11670.69 | 11686.46 | 11702.22 |
| 11719.74 | 11732.72 | 11745.69 |
| 11746.38 | 11756.13 | 11765.89 |
| 11769.80 | 11786.10 | 11802.40 |
| 11826.75 | 11843.48 | 11860.20 |
| 11876.81 | 11889.88 | 11902.95 |
| 11903.39 | 11913.25 | 11923.11 |
| 11927.82 | 11938.26 | 11948.69 |
| 11974.12 | 11997.34 | 12020.56 |
| 12084.48 | 12116.90 | 12149.32 |
| 12151.24 | 12160.63 | 12170.03 |
| 12266.86 | 12290.16 | 12313.47 |
| 12552.61 | 12629.48 | 12706.34 |
| 12723.06 | 12738.33 | 12753.59 |
| 12769.89 | 12789.06 | 12808.24 |
| 12834.49 | 12917.52 | 13000.55 |
| 13018.32 | 13031.40 | 13044.48 |
| 13049.54 | 13076.86 | 13104.18 |
| 13119.56 | 13135.29 | 13151.02 |
| 13265.30 | 13276.12 | 13286.94 |
| 13304.84 | 13325.96 | 13347.09 |
| 13351.99 | 13364.15 | 13376.31 |
| 13501.19 | 13524.33 | 13547.48 |
| 13554.22 | 13569.52 | 13584.82 |
| 13602.38 | 13612.58 | 13622.78 |
| 13708.20 | 13723.60 | 13739.00 |
| 13740.40 | 13762.02 | 13783.64 |
| 13783.92 | 13795.98 | 13808.04 |
| 13832.96 | 13846.00 | 13859.04 |
| 13860.73 | 13881.13 | 13901.52 |
| 13905.76 | 13917.74 | 13929.71 |
| 13929.96 | 13944.37 | 13958.78 |
| 13959.98 | 13981.28 | 14002.58 |
| 14014.11 | 14067.59 | 14121.06 |
| 14122.86 | 14174.53 | 14226.20 |
| 14229.93 | 14254.82 | 14279.70 |
| 14280.60 | 14301.90 | 14323.20 |
| 14401.51 | 14431.22 | 14460.94 |
| 14462.27 | 14541.41 | 14620.56 |
| 14623.06 | 14642.87 | 14662.69 |
| 14684.56 | 14699.66 | 14714.76 |
| 14764.89 | 14786.87 | 14808.84 |
| 14859.96 | 14882.15 | 14904.35 |
| *15114.72 | 15136.98 | 15159.25 |
| *15321.76 | 15344.32 | 15366.88 |
| *15856.28 | 15879.62 | 15902.96 |
| *16066.16 | 16087.64 | 16109.12 |
| 18248.47 | 18271.03 | 18293.59 |
| 18548.49 | 18570.16 | 18591.84 |
| 18603.02 | 18630.68 | 18658.34 |
| 18708.84 | 18730.03 | 18751.21 |
| 18811.43 | 18848.65 | 18885.87 |
| 19343.00 | 19378.06 | 19413.11 |
| 20886.02 | 21156.71 | 21427.39 |
| 21669.84 | 21804.69 | 21939.55 |
| 22566.70 | 22604.25 | 22641.79 |
| 22999.81 | 23033.14 | 23066.47 |
| 23097.51 | 23130.26 | 23163.02 |
| 23213.01 | 23246.92 | 23280.82 |
| 23305.86 | 23353.04 | 23400.22 |
| 23429.20 | 23467.05 | 23504.91 |
| 25144.70 | 25185.27 | 25225.84 |
| 25429.35 | 25473.61 | 25517.87 |
| 25519.61 | 25570.37 | 25621.14 |
| 25624.40 | 25686.01 | 25747.63 |
| 27915.48 | 27962.78 | 28010.08 |
| 28037.85 | 28133.53 | 28229.20 |
| 28237.01 | 28338.55 | 28440.09 |
| 28800.67 | 28859.90 | 28919.13 |
| 28924.34 | 28972.72 | 29021.10 |
| 29030.65 | 29078.60 | 29126.55 |

We claim:

1. A method for detecting a class label in an early stage non-small-cell lung cancer patient needing surgery to treat the cancer, comprising the steps of:
(a) conducting mass spectrometry on a blood-based sample obtained from the patient and obtaining integrated intensity values in the mass spectral data of a multitude of pre-determined mass-spectral features, and
(b) operating on the mass spectral data with a programmed computer implementing a classifier, wherein the programmed computer performs a hierarchical classification procedure on the mass spectrometry data, including a first classifier (Classifier A) producing a class label in the form of high risk or low risk or the equivalent, and if the Classifier A produces the high risk label the sample is classified by a second classifier (Classifier B) generating a classification label of highest risk or high/intermediate risk or the equivalent, and
wherein in the operating step the classifier compares the integrated intensity values obtained in step (a) with feature values of a reference set of class-labeled mass spectral data obtained from blood-based samples obtained from a multitude of other early stage non-small-cell lung cancer patients with a classification algorithm and detects a class label for the sample in accordance with the hierarchical classification schema relating to the risk of the cancer recurring in said patient after surgery.

2. The method of claim 1, wherein the programmed computer stores a reference set of mass spectrometry data used for classification by classifiers A and B obtained from blood-based samples obtained from a multitude of early stage non-small-cell cancer patients, and wherein the mass spectrometry data includes integrated intensity values for features listed in Appendix A.

3. The method of claim 1, wherein the programmed computer implements a hierarchical classifier schema including a third classifier (Classifier C) wherein if the classifier A produces a “low risk” classification label the sample is classified by the third classifier C and wherein classifier C produces a class label of lowest risk or low/intermediate risk or the equivalent.

4. The method of claim 3, wherein classifiers A, B and C are combined in a four-way hierarchical schema as shown in FIG. 3.

5. The method of claim 3, wherein classifiers A, B and C are combined in a three-way hierarchical schema as shown in FIG. 14.

6. The method of claim 4, wherein each of the classifiers A, B and C comprise a combination of a multitude of master classifiers each developed from a different separation of a development sample set used to generate classifiers A, B and C into training and test sets.

7. The method of claim 1, wherein the blood-based sample is obtained before surgery to treat the cancer.

8. The method of claim 1, wherein the blood-based sample is obtained after surgery to treat the cancer and wherein the reference set of class-labeled mass spectral data obtained from blood-based samples obtained from a multitude of other early stage non-small-cell lung cancer patients after surgery to treat the cancer.

9. The method of claim 1, further comprising performing steps (a) and (b) on blood-based samples of the patient obtained before and after surgery to treat the cancer.

10. A method for performing a risk assessment of recurrence of cancer in an early stage non-small-cell lung cancer patient; comprising the steps of:
performing mass spectrometry on a blood-based sample obtained from the patient and obtaining mass spectrometry data, and
in a programmed computer, performing a hierarchical classification procedure on the mass spectrometry data wherein the computing machine implements a hierarchical classifier schema including a first classifier (Classifier A) producing a class label in the form of high risk or low risk or the equivalent, and if the Classifier A produces the high risk label the sample is classified by a second classifier (Classifier B) generating a classification label of highest risk or high/intermediate risk or the equivalent, wherein if Classifier B produces the label of highest risk or the equivalent the patient is predicted to have a high risk of recurrence of the cancer following surgery.

11. The method of claim 10, wherein the programmed computer stores a reference set of mass spectrometry data used for classification by classifiers A and B obtained from blood-based samples obtained from a multitude of early stage non-small-cell cancer patients, and wherein the mass spectrometry data includes feature values for features listed in Appendix A.

12. The method of claim 10, wherein the computing machine implements a hierarchical classifier schema including a third classifier (Classifier C) wherein if the classifier A produces a “low risk” classification label the sample is classified by the third classifier C and wherein classifier C produces a class label of lowest risk or low/intermediate risk or the equivalent.

13. The method of claim 12, wherein classifiers A, B and C are combined in a four-way hierarchical schema as shown in FIG. 3.

14. The method of claim 13, wherein classifiers A, B and C are combined in a three-way hierarchical schema as shown in FIG. 14.

15. The method of claim 13, wherein each of the classifiers A, B and C comprise a combination of a multitude of master classifiers each developed from a different separation of a development sample set used to generate classifiers A, B and C into training and test sets.

16. A programmed computer making a prediction of the risk of recurrence of cancer in an early stage non-small-cell lung cancer patient from a blood-based sample obtained from the patient, comprising a processing unit and a memory storing code and classifier parameters such that the computer is configured as a hierarchical classifier as per FIG. 3 or FIG. 14 combining classifiers A, B and C, the memory further storing a reference set of mass spectral data from blood-based samples obtained from a multitude of early stage non-small cell lung cancer patients for use in classification of the blood-based sample including feature values of the features listed in Appendix A.

17. The programmed computer of claim 16, wherein:
Classifier A is defined by parameters such that it generates a class label of high risk or the equivalent and low risk or the equivalent;
Classifier B is used to classify a sample previously classified as high risk or the equivalent by Classifier A, and is defined by parameters such that it generates a class label of highest risk or the equivalent and an intermediate classification or the equivalent; and wherein
Classifier C is used to classify a sample previously classified as low risk or the equivalent by Classifier A, and is defined by parameters such that it generates a class label of lowest risk or the equivalent and an intermediate classification or the equivalent.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The method of claim 1, wherein the blood-based sample obtained from the patient is a pre-surgery blood-based sample, wherein the integrated intensity values in the mass spectral data of a multitude of pre-determined mass-spectral features are as listed in Appendix A, wherein:
(1) the mass spectrum of the sample is classified with a computer-based classifier developed from a set of blood-based samples obtained from other early stage NSCLC patients, the classifier producing a label of high or highest risk of recurrence or the equivalent and low or lowest risk of recurrence or the equivalent;
(2) wherein, if the sample is not classified as high or highest risk of recurrence in accordance with the classification produced in step (1), obtaining a further blood-based sample from the patient after the surgery and conducting mass spectrometry on the blood-based sample including obtaining integrated intensity values of the features listed in Appendix A; and
(3) classifying the mass spectrum of the sample obtained in (2) in accordance with a computer-based classifier developed from a set of blood-based samples obtained from other early stage NSCLC patients after surgery, wherein the classifier (3) generates a class label of either G1 or the equivalent or G2 or the equivalent, with G2 class label associated with a prediction that the patient will have a lower risk of recurrence as compared to risk of recurrence associated with the class label G1.

32. The method of claim 31, further comprising guiding treatment of patients based on the class label developed in (3).

33. A method for guiding treatment of an early stage non-small-cell lung cancer patient comprising:
(A) detecting a class label in the patient comprising the steps of:
(i) conducting mass spectrometry on a pre-surgery blood-based sample obtained from the patient and obtaining integrated intensity values in the mass spectral data of a multitude of pre-determined mass-spectral features shown in Appendix A, wherein the mass spectrum of the sample is classified with a computer-based classifier developed from a set of blood-based samples obtained from other early stage NSCLC patients, the classifier producing a label of high or highest risk of recurrence or the equivalent and low or lowest risk of recurrence or the equivalent;
(ii) wherein, if the sample is not classified as high or highest risk of recurrence in accordance with the classification produced in step (i), obtaining a further blood-based sample from the patient after the surgery and conducting mass spectrometry on the blood-based sample including obtaining integrated intensity values of the features listed in Appendix A; and
(iii) classifying the mass spectrum of the sample obtained in (ii) in accordance with a computer-based classifier developed from a set of blood-based samples obtained from other early stage NSCLC patients after surgery, wherein the classifier (iii) generates a class label of either G1 or the equivalent or G2 or the equivalent, with G2 class label associated with a prediction that the patient will have a lower risk of recurrence as compared to risk of recurrence associated with the class label G1; and
(B) guiding treatment of the patient based on the class label developed in step (A)(iii).

34. The method of claim 33, wherein the treatment based on the class label includes adjuvant chemotherapy, radiation therapy, immunotherapy, radiotherapy or more close follow-up and observation.
